(19) United States
(12) Patent Application Publication
(10) Pub. No.: US 2026/0363997 A1
(43) Pub. Date: Nov. 19, 2026

(54) METHOD FOR TREATING HAIR

(71) Applicant: KAO CORPORATION, Tokyo (JP)

(72) Inventors: Michael MOLENDA, Sumida-ku (JP); Laila TEMMA, Sumida-ku (JP); Eisuke MIYOSHI, Sumida-ku (JP)

(73) Assignee: KAO CORPORATION, Tokyo (JP)

(21) Appl. No.: 19/102,186

(22) PCT Filed: Aug. 12, 2022
(86) PCT No.: PCT/JP2022/030808

§ 371 Date: Feb. 7, 2025

(51) Int. Cl. A61K 8/81; A61K 8/73; A61K 8/891; A61K 8/898; A61Q 5/12

(52) U.S. Cl. CPC A61K 8/8158; A61K 8/731; A61K 8/733; A61K 8/8152; A61K 8/8176; A61K 8/891; A61K 8/898; A61Q 5/12; A61K 2800/884

(57) ABSTRACT

A method of treating hair that involves a step 1 of applying a composition I to the hair and a step 2 of applying a composition II to the hair. Step 1 and step 2 may be performed in any order, but the hair is not rinsed with water between step 1 and step 2. Composition I includes the components (A): a cationic polymer having a viscosity of 1,000 mPa-s or more at 30 °C in a 1% by mass aqueous solution and (B): an anionic polymer. Composition II includes the components (C): one or more modified silicones selected from the group consisting of polyglycerin-modified silicones and amino-modified silicones; and (D): dimethylpolysiloxane.

### TECHNICAL FIELD

The present invention relates to a method of treating hair and a kit for treating hair.

### BACKGROUND ART

Various methods have been proposed to improve hair texture.

For example, International Publication No. 2010/141683 (Patent Literature 1) discloses, as a method of treating hair with a composite product system that provides an improved conditioning effect on hair, a composite product system for keratin materials, including: applying to the hair a predetermined amount of a hair cleaning composition including a carrier, a specific surfactant, and a predetermined amount of a silicone emulsion; and applying to the hair a predetermined amount of a hair conditioning composition containing a cationic surfactant, a high melting point aliphatic compound, and an aqueous carrier and having predetermined physical properties.

International Publication No. 2017/151995 (Patent Literature 2) discloses a method for treating hair to provide an improved conditioning effect, such as a feeling of hair manageability and/or a reduction of frizz or friction, the method including: a) a step of applying a shampoo composition containing a detergent surfactant to the hair, wherein the shampoo composition is substantially free of silicone conditioning agents; b) a step of rinsing the shampoo composition from the hair; and c) a step of applying to the hair a conditioning composition containing a silicone resin and an amino silicone, wherein the amino silicone has a non-quaternized and/or quaternary amine functional group.

International Publication No. 2016/151139 (Patent Literature 3) discloses a method of cosmetically treating hair for care and/or cleansing, the method including: a step (i) of applying to the hair a first cosmetic composition containing a cationic surfactant, a silicone, and a non-silicone fatty substance; a step (ii) of applying to the hair a second cosmetic composition containing a cationic surfactant, a predetermined cationic polymer, and an organosilane; and not including an intermediate rinsing process between the application steps (i) and (ii).

International Publication No. 2020/146931 (Patent Literature 4) discloses a hair care system that includes a specific shampoo composition and a conditioner composition.

### SUMMARY OF INVENTION

The present invention relates to a method of treating hair, including the following step 1 and step 2 in any order and not including rinsing the hair with water between step 1 and step 2:
Step 1: a step of applying composition I containing the following component (A) and component (B) to the hair;
Component (A): a cationic polymer having a viscosity of 1,000 mPa-s or more at 30 °C in a 1% by mass aqueous solution;
Component (B): an anionic polymer;
Step 2: a step of applying composition II containing the following component (C) and component (D) to the hair;
Component (C): one or more modified silicones selected from the group consisting of polyglycerin-modified silicones and amino-modified silicones;
Component (D): a dimethylpolysiloxane.

### DESCRIPTION OF EMBODIMENTS

Hair problems includes problems of hair shape, such as frizz, spreading, waviness, wavy hair, and splashing and the problems of hair texture such as dryness, and a hair treatment method is required to improve these problems.

In conventional hair treatment methods, regarding the hair to be treated, smoothness and moistness have a trade-off relationship with lightness and naturalness, and it has been difficult to obtain hair with all items excellent. It has been also particularly difficult to achieve excellent texture independent of the diameter of the hair and the degree of damage. For example, formulations designed for thick hair tend to provide a heavy feel when applied to fine hair due to excessive silicone components adhering to the hair. In addition, the hair surface is covered with a cuticle and is hydrophobic; however, it is known that excessive washing or brushing of the hair, or chemical treatments such as perms and bleaching can cause the cuticle to deteriorate or peel off, resulting in the hair surface becoming hydrophilic. Therefore, formulations designed for hair with a high degree of damage tend to provide a heavy feel on hair with a low degree of damage because excessive amounts of hydrophobic feel enhancers such as silicones adhere on the hair.

The technology described in Patent Literature 1 is a formulation that is substantially free of anionic polymers and is insufficient in terms of hair wash resistance and moistness.

The technology described in Patent Literature 2 is a formulation that is free of cationic polymers and anionic polymers in the same composition, and is normal shampoo and treatment compositions that use MQ resin as a substitute for a silicone compound, and is insufficient in terms of hair wash resistance and weightless finish.

The technology described in Patent Literature 3 is a formulation that is free of cationic polymers and anionic polymers in the same composition, as is the case with the technology described in Patent Literature 2.

The present invention relates to a method of treating hair, being capable of achieving the effect of improving the shape and texture of hair and the sustainability of this effect, not depending on the diameter of the hair and the degree of damage. Particularly, the present invention relates to a method of treating hair, being capable of simultaneously achieving smoothness and moistness and lightness and naturalness, which are conventionally difficult to be simultaneously achieved.

The present inventors have found to be able to provide a method of treating hair, being capable of achieving the effect of improving the shape and texture of hair and the sustainability of this effect, not depending on the diameter of the hair and the degree of damage, and being capable of simultaneously achieving the smoothness and moistness and lightness and naturalness, by the method of treating hair, including two steps in any order: a step 1 of applying the predetermined composition I to the hair; and a step 2 of applying the predetermined composition II to the hair, and not including rinsing the hair with water between step 1 and step 2.

The present invention includes the following method of treating hair and kit for treating hair:
[1] A method of treating hair, including the following step 1 and step 2 in any order and not including rinsing the hair with water between step 1 and step 2:
Step 1: a step of applying composition I containing the following component (A) and component (B) to the hair;
Component (A): a cationic polymer having a viscosity of 1,000 mPa-s or more at 30 °C in a 1% by mass aqueous solution;
Component (B): an anionic polymer;
Step 2: a step of applying composition II containing the following component (C) and component (D) to the hair;
Component (C): one or more modified silicones selected from the group consisting of polyglycerin-modified silicones and amino-modified silicones;
Component (D): a dimethylpolysiloxane.
[2] A kit for treating hair, including composition I containing the following component (A) and component (B) for application to hair, and composition II containing the following component (C) and component (D) for application to hair.
Component (A): cationic polymer having a viscosity of 1,000 mPa-s or more at 30 °C in a 1% by mass aqueous solution;
Component (B): anionic polymer;
Component (C): one or more modified silicones selected from the group consisting of polyglycerin-modified silicone and amino-modified silicone; and
Component (D): dimethylpolysiloxane.

The present invention provides a method of treating hair, being capable of achieving the effect of improving the shape and texture of hair and the sustainability of this effect, not depending on the diameter of the hair and the degree of damage, and being capable of simultaneously achieving smoothness and moistness and lightness and naturalness.

### <Method of Treating Hair>

The method of treating hair according to the present invention includes the following step 1 and step 2 in any order and does not include rinsing the hair with water between step 1 and step 2:
Step 1: a step of applying composition I containing the following component (A) and component (B) to the hair;
Component (A): a cationic polymer having a viscosity of 1,000 mPa-s or more at 30 °C in a 1% by mass aqueous solution;
Component (B): an anionic polymer;
Step 2: a step of applying composition II containing the following component (C) and component (D);
Component (C): one or more modified silicones selected from the group consisting of polyglycerin-modified silicones and amino-modified silicones;
Component (D): a dimethylpolysiloxane.

In this description, “containing component X” also includes formulating component X.

The method of treating hair according to the present invention has been found to be capable of achieving the effect of improving the shape and texture of hair and the sustainability of this effect, not depending on the diameter of the hair and the degree of damage, and to be capable of simultaneously achieving smoothness and moistness and lightness and naturalness, by the method of treating hair, including the above step 1 and step 2.

It is not certain why the method of treating hair according to the present invention achieves the above effect; however, the following is assumed to be the reason.

Composition I contains component (A), a predetermined cationic polymer, and component (B), an anionic polymer, forms on the hair a water-insoluble film consisting of a so-called polyion complex in which a cross-linked structure is formed by ionic interaction between component (A) and component (B). The film formed by composition I is presumed to condition the shape of the hair.

In addition, composition II containing component (C), a predetermined modified silicone, and component (D), a dimethylpolysiloxane, is presumed to form a persistent hydrophobic film and to condition the texture (feel) of the hair.

Furthermore, the film formed by composition II after the hair was treated with composition I is assumed to simultaneously achieve lightness and naturalness, improved smoothness and moistness without overloading. The reason for this combined effect is that the polyion complex formed by composition I can have a polar interaction with the modified silicone and a hydrophobic interaction with the dimethylpolysiloxane from composition II. By this, the uptake amount is adjusted independent from diameter of the hair and degree of damage. Particularly, by not rinsing the hair with water between step 1 and step 2, it is assumed that no excessive swelling of the polyion complex formed by composition I happens. Without excessive swelling, polar interaction is not disturbed between the polyion complex formed by composition I and the modified silicone in the film formed by composition II. By this, the effect of the present invention occurs on hair.

The method of treating hair according to the present invention includes the above step 1 and step 2 in any order and does not include the step of rinsing the hair with water between step 1 and step 2.

Steps other than rinsing the hair with water may or may not be included between step 1 and step 2. From the viewpoint of the effect of the present invention, step 1 and step 2 are preferably performed consecutively. “Performing consecutively” means that one step is performed immediately after another step, without the step of rinsing the hair with water and without the step other than rinsing. Preferably, composition I and composition II are applied to the hair without any other steps therebetween.

In one of the preferred aspects of the present invention, step 1 and step 2 are performed in sequence, from the viewpoint of ease of conditioning the shape of the object to be treated and improving the sustainability thereof, particularly, the effect of suppressing the spread of the hair and the sustainability thereof, regardless of the diameter of the hair and the degree of damage. In this case, step 1 applies composition I directly to the hair and does not include the step of rinsing the hair with water between step 1 and step 2, and step 2 applies composition II to the hair to which composition I is applied.

In such an aspect, the polyion complex formed by composition I is first adsorbed by the hair, and then the modified silicone in composition II forms polar interaction and dimethylpolysiloxane forms hydrophobic interaction, respectively, with the polyion complex formed by the above composition I therebetween. It is assumed that each uptake amount is adjusted not depending on the diameter of the hair and the degree of damage, thereby not only improving smoothness and moistness but also simultaneously achieving lightness and naturalness due to no overloading.

In addition, prior to steps 1 and 2, any one or more steps selected from the following step A and step B can be performed.

Step A: a step of applying a shampoo to the hair and optionally rinsing the shampooed hair,

Step B: a step of applying a conditioner to the hair and optionally rinsing the conditioner-applied hair.

After performing step 1 and step 2, it is preferable to perform a post-step in which composition I and composition II applied to the hair are rinsed with water. In this case, in order to fully apply composition I and composition II to the hair, a leaving time may be set after performing step 1 and step 2 before rinsing with water. The leaving time is preferably 15 seconds or more, more preferably 30 seconds or more, more preferably 1 minute or more, and preferably 20 minutes or less, more preferably 15 minutes or less.

The time for rinsing composition I and composition II with water is preferably 5 seconds or more and 3 minutes or less. The temperature of water may be just enough not to strain the body, preferably 15 °C or more and 50 °C or less, more preferably 25 °C or more and 45 °C or less.

The hair can then optionally be subjected to drying with, for example, a towel and a hairdryer or to heating with, for example, an iron.

In addition, hair styling agents can optionally be applied to style the hair into various forms.

{Step 1}

Step 1 is a step of applying composition I containing the above component (A) and component (B) to the hair.

Specific product forms of composition I include hair shampoos, hair rinses, hair conditioners, hair treatments (including non-rinsing types), hair styling agents, hair colors, and permanent care products. Among these, from the viewpoint of effectiveness of the effect of the present invention, hair conditioners, hair treatments, or hair styling agents are preferable.

The form of composition I is preferably a liquid or gel composition, and more preferably a liquid composition from the viewpoint of improving the stability of the composition and the feel of use.

The term “liquid or gel” in the present invention means that one is determined to be liquid in the liquid-solid determination test according to the American Society for Testing and Materials standard “ASTM D 4359-90: Standard Test Method for Determining Whether a Material is a Liquid or Solid”.

Hereinafter, each component contained in composition I is explained.

### (Component (A): Cationic Polymer)

Component (A) is a cationic polymer having a viscosity of 1,000 mPa-s or more at 30 °C in a 1% by mass aqueous solution, and is a polymer that can form a polyion complex through interaction with component (B).

Component (A), the cationic polymer, is preferably a polymer that has cationic groups and is positively charged as a total charge. Component (A) may have anionic groups, nonionic groups, or amphoteric groups such as betaine groups in addition to cationic groups to the extent that it does not interfere with the effect of the present invention.

In the present description, a cationic group is a cationic group or a group that can be ionized to become a cationic group, specifically a primary amino group, secondary amino group, tertiary amino group, and quaternary ammonium group.

Anionic groups are anion groups or groups that can be ionized to become anion groups, specifically one or more selected from the group consisting of acidic groups such as carboxy groups, sulfonic acid groups, and phosphoric acid groups, preferably one or more selected from the group consisting of carboxy groups and sulfonic acid groups, more preferably a carboxy group. At least a part of the anionic groups may be neutralized to become a salt state.

From the viewpoint of ease of forming a polyion complex, ease of conditioning the shape of the object to be treated, and improvement of the sustainability thereof, particularly from the viewpoint of the effect of suppressing the spread of the hair and improving the sustainability thereof, and from the viewpoint of improving the stability of the composition, the viscosity of a 1% by mass aqueous solution of component (A) at 30 °C is 1,000 mPa-s or more, preferably 1,300 mPa-s or more, more preferably 1,500 mPa-s or more, further preferably 1,700 mPa-s or more, and still more preferably 1,800 mPa-s or more. The upper limit of the viscosity is not particularly limited; however, from the viewpoint of ease of forming a polyion complex and handling, the viscosity is preferably 100,000 mPa-s or less, more preferably 80,000 mPa-s or less, further preferably 50,000 mPa-s or less, still more preferably 30,000 mPa-s or less, and still more preferably 25,000 mPa-s or less. The viscosity of a 1% by mass aqueous solution of component (A) at 30 °C is 1,000 mPa-s or more, preferably 1,000 mPa-s or more and 100,000 mPa-s or less, more preferably 1,300 mPa-s or more and 80,000 mPa-s or less, further preferably 1,500 mPa-s or more and 50,000 mPa-s or less, still more preferably 1,700 mPa-s or more and 30,000 mPa-s or less, and still more preferably 1,800 mPa-s or more and 25,000 mPa-s or less.

The viscosity of a 1% by mass aqueous solution of component (A) at 30 °C can be measured by the method specifically described in Examples.

The unit of viscosity is “mPa-s” and is an abbreviation of “millipascal second”.

The weight average molecular weight (Mw) of component (A) is preferably 5,000 or more, more preferably 10,000 or more, further preferably 30,000 or more, still more preferably 50,000 or more, and still more preferably 100,000 or more, from the viewpoint of improving film forming ability. The upper limit of the molecular weight is not particularly limited and is preferably 3,000,000 or less, more preferably 2,000,000 or less, and more preferably 1,500,000 or less, from the viewpoint of ease of forming a polyion complex and handling. The weight average molecular weight (Mw) of component (A) is preferably 5,000 or more and 3,000,000 or less, more preferably 10,000 or more and 2,000,000 or less, further preferably 30,000 or more and 2,000,000 or less, still more preferably 50,000 or more and 1,500,000 or less, and still more preferably 100,000 or more and 1,500,000 or less.

The weight average molecular weight of component (A) can be measured by gel permeation chromatography (GPC).

The cation charge density of component (A) is preferably 0.1 mmol/g or more, more preferably 0.2 mmol/g or more, further preferably 0.3 mmol/g or more, and still more preferably 0.5 mmol/g or more, from the viewpoint of ease of forming a polyion complex through interaction with component (B) and the viewpoint of improving the stability of the composition. In addition, from the viewpoint of interaction with the film formed by composition II and from the viewpoint of improving the stability of the composition, the cation charge density of component (A) is preferably 20 mmol/g or less, more preferably 15 mmol/g or less, further preferably 12 mmol/g or less, still more preferably 10 mmol/g or less, still more preferably 7.0 mmol/g or less, and still more preferably 5.0 mmol/g or less. The cation charge density of component (A) is preferably 0.1 mmol/g or more and 20 mmol/g or less, more preferably 0.2 mmol/g or more and 15 mmol/g or less, further preferably 0.2 mmol/g or more and 12 mmol/g or less, still more preferably 0.3 mmol/g or more and 10 mmol/g or less, still more preferably 0.3 mmol/g or more and 7.0 mmol/g or less, and still more preferably 0.5 mmol/g or more and 5.0 mmol/g or less.

The cation charge density of component (A) is the number of moles of cationic groups included in 1 g of the polymer. When at least a part of the cationic groups of component (A) becomes neutralized salts, the number of moles of cationic groups in the salt state shall be included in the number of moles of cationic groups above.

Two or more polymers may be used as component (A), and in this case, the cation charge density of component (A) is determined by calculating the weighted average from the cation charge density and formulated amount of each polymer.

As component (A), any of natural polymers such as cationic polysaccharides or the cationic derivatives thereof, and synthetic polymers can be used, and from the viewpoint of ease of availability, synthetic polymers are preferable.

Specific examples of the polymer used as component (A) include cationated polyvinyl alcohol, polyethyleneimine, methacryloyl ethyltrimethylammonium salt polymer, quaternized dialkylaminoalkyl (meth)acrylate polymer, diallyl quaternized ammonium salt polymer, methacrylamide propyltrimethylammonium salt polymer, vinylimidazolium trichloride-vinylpyrrolidone copolymer (polyquaternium-16), vinylpyrrolidone-alkylamino (meth)acrylate copolymer, vinylpyrrolidone-alkylamino (meth)acrylate-vinylcaprolactam copolymer, alkylacrylamide(meth)acrylate-alkylaminoalkylacrylamide-polyethylene glycol (meth)acrylate copolymer, adipate-dimethylaminohydroxypropylethylenetriamine copolymer, all of which have the viscosity of 1,000 mPa-s or more at 30 °C in a 1% by mass aqueous solution, and one or two or more of these can be used. In the present description, “(meth)acrylic acid” means acrylic acid or methacrylic acid, and “(meth)acrylate” means acrylate or methacrylate.

Among these, examples of the methacryloyl ethyltrimethylammonium salt polymer include methacryloyl ethyl trimethylammonium chloride polymer (polyquaternium-37), methacryloylethyldimethylbetaine-methacryloylethyltrimethylammonium chloride-methoxypolyethylene glycol methacrylate copolymer (polyquaternium-49), and methacryloylethyl dimethylbetaine-methacryloylethyltrimethylammonium chloride-2-hydroxyethyl methacrylate copolymer (polyquaternium-48).

Examples of the quaternized dialkylaminoalkyl (meth)acrylate polymer include vinylpyrrolidone-N,N-dimethylaminoethyl methacrylate diethyl sulfate copolymer (polyquaternium-11), and N,N-dimethylaminoethyl methacrylate diethyl sulfate-N,N-dimethylacrylamide-polyethylene glycol dimethacrylate copolymer (polyquaternium-52).

Examples of the diallyl quaternized ammonium salt polymer include dimethyldiallyl ammonium chloride polymer (polyquaternium-6), dimethyldiallyl ammonium chloride-acrylic acid copolymer (polyquaternium-22), dimethyldiallyl ammonium chloride-acrylamide copolymer (polyquaternium-7), and acrylamide-acrylic acid-dimethyldiallylammonium chloride copolymer (polyquaternium-39).

In addition, examples of the methacrylamide propyltrimethylammonium salt polymer include methacrylamide propyltrimethylammonium chloride polymer, vinylpyrrolidone-methacrylamide propyltrimethylammonium chloride copolymer, acrylic acid-methyl acrylate-methacrylamide propyltrimethylammonium chloride copolymer (polyquaternium-47), and acrylic acid-acrylamide-methacrylamide propyltrimethylammonium chloride copolymer (polyquaternium-53).

Component (A) is preferably a polymer including a constituent unit represented by the following general formula (1), from the viewpoint of improving film forming ability and the viewpoint of ease of forming a polyion complex through interaction with component (B).

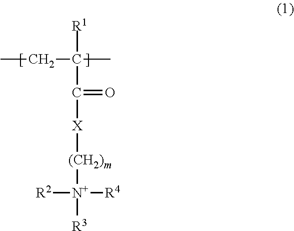

wherein R¹ is a hydrogen atom or a methyl group, R² to R⁴ are each independently an alkyl group having 1 to 4 carbon atoms, X is —O— or —NH—, and m is a number of 1 or more and 4 or less.

R¹ in the general formula (1) is preferably a methyl group, and R² to R⁴ are each independently preferably an alkyl group having 1 to 3 carbon atoms, more preferably a methyl group or ethyl group, and further preferably a methyl group.

X in the general formula (1) is preferably —O— and m is preferably 1 or more and 3 or less, and more preferably 2 or more and 3 or less.

Examples of the constituent unit represented by the general formula (1) include one or more selected from the group consisting of a constituent unit derived from methacryloylethyltrimethylammonium salt, a constituent unit derived from N,N-dimethylaminoethyl methacrylic acid diethyl sulfate, and methacrylamide propyltrimethylammonium salt, and preferably one or more selected from the group consisting of a constituent unit derived from methacryloylethyltrimethylammonium salt and a constituent unit derived from N,N-dimethylaminoethyl methacrylate diethyl sulfate.

Component (A) may be a polymer containing other constituent units other than the constituent unit represented by the general formula (1). Examples of other constituent units include constituent units derived from: vinyl monomers such as vinylpyrrolidone; amphoteric monomers such as (meth)acryloyl ethyldimethyl betaine; hydroxyalkyl (meth)acrylates such as (meth)acrylic acid alkyl esters and (meth)acrylic acid 2-hydroxyethyl; and acrylamides such as N,N-dimethylacrylamide. In addition, component (A) may be a cross-polymer crosslinked with, for example, di(meth)acrylic acid polyethylene glycol.

Component (A) may be used singly or in combination of two or more. From the viewpoint of ease of setting the viscosity of a 1% by mass aqueous solution at 30 °C to a predetermined value or more, and the viewpoints of ease of forming a polyion complex, ease of conditioning the shape of the object to be treated and the improvement of the sustainability thereof, particularly, the effect of suppressing the spread of the hair and improving the sustainability thereof, among these, component (A) is: preferably one or more selected from the group consisting of a methacryloylethyltrimethylammonium salt polymer and a quaternized dialkylaminoalkyl (meth)acrylate polymer; more preferably one or more selected from the group consisting of a methacryloyl ethyl trimethylammonium chloride polymer (polyquaternium-37), a methacryloylethyldimethylbetaine-methacryloylethyltrimethylammonium chloride-methoxypolyethylene glycol methacrylate copolymer (polyquaternium-49), a methacryloylethyldimethylbetaine-methacryloylethyltrimethylammonium chloride-2-hydroxyethyl methacrylate copolymer (polyquaternium-48), and an N,N-dimethylaminoethyl methacrylate diethyl sulfate-N,N-dimethylacrylamide-polyethylene glycol dimethacrylate copolymer (polyquaternium-52); further preferably one or more selected from the group consisting of a methacryloyl ethyl trimethylammonium polymer (polyquaternium-37) and an N,N-dimethylaminoethyl methacrylate diethyl sulfate-N,N-dimethylacrylamide-polyethylene glycol dimethacrylate copolymer (polyquaternium-52).

A commercially available polymer can also be used as component (A). Specific examples of the commercially available polymer include “Cosmedia Ultragel 300” manufactured by BASF Japan Ltd. (polyquaternium-37, cation charge density: 4.81 mmol/g), and “SOFCARE KG-101W-E” (polyquaternium-52, cation charge density: 0.83 mmol/g) and “SOFCARE KG-301W” (polyquaternium-52, cation charge density: 1.84 mmol/g) manufactured by Kao Corporation

### (Component (B): Anionic Polymer)

Component (B) is a polymer capable of forming a polyion complex by interacting with component (A). As used herein, the term “anionic polymer” means a polymer having an anionic group and substantially free of a cationic group and an amphoteric group. “Substantially free of a cationic group and an amphoteric group” means that the molar amount of the cationic group and the amphoteric group with respect to the anionic group is preferably 0.1% or less.

As the anionic group included in component (B), an acidic group such as a carboxy group, a sulfonic acid group, and a phosphoric acid group is preferable, and a carboxy group is more preferable, from the viewpoint of ease of forming a polyion complex by interaction with the component (A), and the viewpoint of availability. At least a part of the anionic groups in component (B) may be neutralized to become a salt state.

From the viewpoint of ease of forming a polyion complex, ease of conditioning the shape of the object to be treated and improvement of the sustainability thereof, particularly from the viewpoint of the effect of suppressing the spread of the hair and its sustainability, and the viewpoint of improving the stability of the composition, component (B) preferably contains a crosslinked polymer containing a constituent unit derived from (meth)acrylic acid, and one or more anionic polymer selected from the group consisting of anionic polysaccharides.

Among the components (B), examples of a crosslinked polymer containing a constituent unit derived from (meth)acrylic acid include a homopolymer or copolymer of (meth)acrylic acid having a crosslinked structure, or a salt thereof. The crosslinked structure referred to herein means a three-dimensional network structure in which polymer chains, the main structures of the polymer, are connected within the polymer chains or between the polymer chains. The method of forming the crosslinked structure is not particularly limited, and examples thereof include the crosslinked structure formed by a method of cross-linking at the same time as polymerization such as polycondensation and radical polymerization, and a method of cross-linking a polymer chain later.

Examples of the homopolymer of the (meth)acrylic acid include polyacrylic acid, polymethacrylic acid, and salts thereof.

Examples of the copolymer of the (meth)acrylic acid include (meth)acrylic acid-maleic acid copolymer, (meth)acrylic acid-itaconic acid copolymer, (meth)acrylic acid-fumaric acid copolymer, (meth)acrylic acid-vinyl acetate copolymer, (meth)acrylic acid-(meth)acrylic acid alkyl ester copolymer, (meth)acrylic acid-2-hydroxyethyl methacrylate copolymer, acrylic acid-acrylic acid alkyl ester-(N-alkyl) acrylamide copolymer, and the salts thereof, and one or two or more of these can be used.

Among these, as a copolymer of (meth)acrylic acid, (meth)acrylic acid-(meth)acrylic acid alkyl ester is preferable, and acrylic acid-acrylic acid alkyl ester is more preferable. Examples of the (meth)acrylic acid alkyl ester include (meth)acrylic acid alkyl ester having an alkyl with preferably 1 or more carbon atoms, more preferably 4 or more, further preferably 8 or more, and preferably 40 or less, more preferably 36 or less, further preferably 32 or less.

Specific examples of the crosslinked polymer containing a constituent unit derived from (meth)acrylic acid include carboxyvinyl polymer or a salt thereof, (acrylate-alkyl acrylate (C10-30)) cross polymer, (Na acrylate-acryloyl dimethyl taurine-dimethyl acrylamide) cross polymer, and acrylate cross polymer-4.

Among components (B), examples of the anionic polysaccharide include polysaccharides having a carboxy group (for example, hyaluronic acid, alginic acid, pectic acid, carboxymethyl cellulose, xanthan gum), polysaccharide sulfates (for example, carrageenan, keratan sulfate, dermatan sulfate, sulfated starch, heparin, heparan sulfate), and the salts thereof, and one or two or more of these can be used.

Among these, from the viewpoint of ease of forming a polyion complex, ease of conditioning the shape of the object to be treated, and improvement of the sustainability thereof, the anionic polysaccharide is preferably a polysaccharide having a carboxy group or a salt thereof, more preferably alginic acid, carboxymethyl cellulose, or a salt thereof, and further preferably alginic acid or a salt thereof.

When component (B) is a salt, examples of the salt include alkali metal salts such as sodium salt and potassium salt, and alkaline earth metal salts, and from the viewpoint of availability, alkali metal salts are preferable.

Among these, from the viewpoint of ease of forming a polyion complex and improvement of the sustainability thereof, component (B) is preferably one or more selected from the group consisting of a carboxyvinyl polymer, a (acrylate-alkyl acrylate (C10-30)) cross polymer, and anionic polysaccharides, and more preferably anionic polysaccharides or salts thereof, further preferably alginic acid or a salt thereof.

The anion charge density of component (B) is not particularly limited, and from the viewpoint of ease of forming a polyion complex by interaction with component (A), the viewpoint of the effect of the present invention, and the viewpoint of improving the stability of the composition, the anion charge density of component (B) is preferably 1.0 mmol/g or more, more preferably 2.0 mmol/g or more, further preferably 3.5 mmol/g or more, and from the viewpoint of the effect of improving the texture of the object to be treated and improving the sustainability thereof, particularly, from the viewpoint of the effect of suppressing hair entanglement when used for hair treatment and improving the sustainability thereof, the anion charge density of component (B) is preferably 25 mmol/g or less, more preferably 20 mmol/g or less, further preferably 15 mmol/g or less. The anion charge density of the component (B) is preferably 1.0 mmol/g or more and 25 mmol/or less, more preferably 2.0 mmol/g or more and 20 mmol/g or less, and still more preferably 3.5 mmol/g or more and 15 mmol/g or less.

The anion charge density of component (B) is the number of moles of anionic groups included in 1 g of the polymer. When at least a part of the anionic group of component (B) becomes a neutralized salt, the number of moles of the anionic group shall include the number of moles of the anionic group in the salt state.

Two or more of polymers may be used as component (B), and the anion charge density of component (B) in this case is determined by calculating the weighted average from the anion charge density and the formulation amount of each polymer.

When component (B) is anionic polysaccharides, from the viewpoint of ease of forming a polyion complex by interaction with component (A), ease of conditioning the shape of the object to be treated and the improvement of the sustainability thereof, particularly, the viewpoint of the effect of suppressing the spread of the hair and improving the sustainability thereof, and the viewpoint of improving the stability of the composition, the weight average molecular weight (Mw) of component (B) is, from the viewpoint of improving the film forming ability, preferably 2,000 or more, more preferably 5,000 or more, further preferably more than 10,000, still more preferably 30,000 or more, and still more preferably 50,000 or more. In addition, from the viewpoint of ease of forming a polyion complex by interaction with component (A), ease of conditioning the shape of the object to be treated and the improvement of the sustainability thereof, particularly, the viewpoint of the effect of suppressing the spread of the hair and improving the sustainability thereof, and the viewpoint of improving the stability of the composition, the weight average molecular weight (Mw) of component (B) is preferably 3,500,000 or less, more preferably 3,000,000 or less, and further preferably 2,500,000 or less. The weight average molecular weight (Mw) of the anionic polysaccharides as component (B) is preferably 2,000 or more and 3,500,000 or less, more preferably 5,000 or more and 3,000,000 or less, further preferably more than 10,000 and 3,000,000 or less, still more preferably 30,000 or more and 2,500,000 or less, and still more preferably 50,000 or more and 2,500,000 or less. The weight average molecular weight of component (B) can be measured by gel permeation chromatography (GPC).

From the viewpoint of improving the film forming ability, the viewpoint of ease of forming a polyion complex by interaction with component (A), ease of conditioning the shape of the object to be treated and the improvement of the sustainability thereof, particularly, the effect of suppressing the spread of the hair and the improvement of the sustainability thereof, and the viewpoint of improving the stability of the composition, the content of component (B) in component (B) is preferably 50% by mass or more, more preferably 60% by mass or more, further preferably 70% by mass or more, still more preferably 80% by mass or more, and 100% by mass or less.

### (Component (G): Nonionic Polymer)

From the viewpoint of improving the film forming ability, ease of conditioning the shape of the object to be treated and the improvement of the sustainability thereof, particularly, the effect of suppressing the spread of the hair and the improvement of the sustainability thereof, and the viewpoint of improving the stability of the composition, the composition I preferably further contains (G) a nonionic polymer. It is assumed that use of component (G) causes the rigid crosslinked structure of the polyion complex formed by component (A) and component (B) to be reinforced by composite of component (G) with a long-chain and flexible non-crosslinked structure.

As used herein, the term “nonionic polymer” means a polymer being substantially free of a cationic group, an anionic group, and an amphoteric group, which are an ionic group.

Examples of component (G) include polyalkylene glycol or a derivative thereof, a polymer including a constituent unit derived from vinylpyrrolidone, a polymer including a constituent unit derived from (meth)acrylic acid ester, polyvinyl alcohol, polyacrylamide, and polycaprolactam, and one or two or more of these can be used. Examples of the polyalkylene glycol include a homopolymer or a copolymer of alkylene glycol having an alkylene with 2 or more and 6 or less carbon atoms, preferably 2 or more and 4 or less, and more preferably 2 or more and 3 or less. Specific examples thereof include polyethylene glycol, polypropylene glycol, polytrimethylene ether glycol, polytetramethylene ether glycol, polyethylene glycol-polypropylene glycol copolymer (polyoxyethylene polyoxypropylene glycol), polyethylene glycol-polytrimethylene ether glycol copolymer, polyethylene glycol-polytetramethylene ether glycol copolymer, and polypropylene glycol-polytetramethylene ether glycol copolymer. Among these, from the viewpoint of the effect of improving the texture of the object to be treated and the improvement of the sustainability of the effect, particularly, the viewpoint of the effect of suppressing hair entanglement when used for hair treatment and the improvement of the sustainability thereof, the viewpoint of improving moisture resistance, and the viewpoint of availability, there is preferable one or more selected from the group consisting of polyethylene glycol, polypropylene glycol, and polyethylene glycol-polypropylene glycol copolymer, more preferably one or more selected from the group consisting of polyethylene glycol and polyethylene glycol-polypropylene glycol copolymer.

Examples of the polyalkylene glycol derivative include an end-modified product of the above polyalkylene glycol, such as polyalkylene glycol monoalkyl ether, polyalkylene glycol dialkyl ether, polyalkylene glycol monoaryl ether, polyalkylene glycol diaryl ether, polyalkylene glycol monofatty acid ester, polyalkylene glycol difatty acid ester, and polyalkylene glycol diglycidyl ether. Among these, from the viewpoint of the effect of improving the texture of the object to be treated and the improvement of the sustainability of the effect, particularly the viewpoint of the effect of suppressing hair entanglement when used for hair treatment and the improvement of the sustainability thereof, and the viewpoint of availability, there is preferable one or more selected from the group consisting of polyalkylene glycol monoalkyl ether, polyalkylene glycol dialkyl ether, polyalkylene glycol monofatty acid ester, and polyalkylene glycol difatty acid ester, more preferably one or more selected from the group consisting of polyalkylene glycol monofatty acid ester and polyalkylene glycol difatty acid ester.

The carbon number of the alkyl group in the monoalkyl or dialkyl ether of the polyalkylene glycol and the carbon number of the fatty acid in the monofatty or difatty acid ester of the polyalkylene glycol are preferably 1 or more and 36 or less, more preferably 4 or more and 32 or less, further preferably 8 or more and 24 or less, still more preferably 12 or more and 22 or less. In addition, the carbon number of the aryl group in the monoaryl or diaryl ether of the polyalkylene glycol is preferably 6 or more and 36 or less, more preferably 6 or more and 32 or less, further preferably 6 or more and 24 or less, still more preferably 6 or more and 22 or less.

Specific examples of the polyalkylene glycol derivative include polyethylene glycol monomethyl ether, polyethylene glycol dimethyl ether, polyethylene glycol monobutyl ether, polyethylene glycol dibutyl ether, polyethylene glycol monooctyl ether, polyethylene glycol monodecyl ether, polyethylene glycol monododecyl ether, polyethylene glycol monotridecyl ether, polyethylene glycol-bisphenol A ether, polyethylene glycol dilaurate, polyethylene glycol distearate (polyethylene glycol distearate), polyethylene glycol diglycidyl ether, polyethylene glycol-polypropylene glycol-monomethyl ether, polyethylene glycol-polypropylene glycol-dimethyl ether, polyethylene glycol-polypropylene glycol-monobutyl ether, polyethylene glycol-polypropylene glycol-dibutyl ether, polyethylene glycol-polypropylene glycol-monooctyl ether, polyethylene glycol-polypropylene glycol-monodecyl ether, polyethylene glycol-polypropylene glycol-monododecyl ether, polyethylene glycol-polypropylene glycol-monotridecyl ether, polyethylene glycol-polypropylene glycol-bisphenol A ether, polyethylene glycol-polypropylene glycol-dilaurate, polyethylene glycol distearate-polypropylene glycol, and polyethylene glycol-polypropylene glycol-diglycidyl ether, and one or two or more of these can be used.

Examples of the polymer containing a constituent unit derived from vinylpyrrolidone include a homopolymer of vinylpyrrolidone or a copolymer of vinylpyrrolidone and a nonionic monomer. Specific examples thereof include polyvinylpyrrolidone, vinylpyrrolidone-vinyl acetate copolymer, and vinylpyrrolidone-methacrylamide-vinylimidazole copolymer.

Examples of the polymer containing a constituent unit derived from (meth)acrylic acid ester include a homopolymer of (meth)acrylic acid ester and a copolymer of (meth)acrylic acid ester and a nonionic monomer such as (meth)acrylamide, dimethylacrylamide, vinyl acetate, and vinyl methyl ether. Examples of the (meth)acrylic acid ester include (meth)acrylic acid hydroxyalkyl ester, (meth)acrylic acid alkyl ether, in addition to the above (meth)acrylic acid alkyl ester.

Specific examples of the polymer containing a constituent unit derived from (meth)acrylic acid ester include (dimethylacrylamide-hydroxyethyl acrylate-methoxyethyl acrylate) copolymer and (hydroxyethyl acrylate-methoxyethyl acrylate) copolymer.

Among these, from the viewpoint of improving the film forming ability, ease of conditioning the shape of the object to be treated and the improvement of the sustainability thereof, particularly, the effect of suppressing the spread of the hair and the improvement of the sustainability thereof, the viewpoint of improving the stability of the composition, and the viewpoint of availability, component (G) is preferably one or more selected from the group consisting of polyalkylene glycol or the derivative thereof and a polymer containing a constituent unit derived from vinylpyrrolidone, more preferably one or more selected from the group consisting of polyethylene glycol, polypropylene glycol, polyethylene glycol-polypropylene glycol copolymer, monofatty or difatty acid ester thereof, and polyvinylpyrrolidone, further preferably one or more selected from the group consisting of polyethylene glycol, polyethylene glycol-polypropylene glycol copolymer, monofatty or difatty acid ester thereof, and polyvinylpyrrolidone.

From the viewpoint of improving the stability of the composition and the viewpoint of availability, the weight average molecular weight (Mw) of component (G) is preferably 2,000 or more, more preferably 5,000 or more, further preferably 10,000 or more, and preferably 2,000,000 or less, more preferably 1,500,000 or less. The weight average molecular weight of component (G) is preferably 2,000 or more and 2,000,000 or less, more preferably 5,000 or more and 1,500,000 or less, and further preferably 10,000 or more and 1,500,000 or less.

The weight average molecular weight of component (G) can be measured by gel permeation chromatography (GPC).

In addition, from the viewpoint of ease of conditioning the shape of the object to be treated and the improvement of the sustainability thereof, particularly, the viewpoint of maintaining the function such as the effect of suppressing the spread of the hair and the improvement of the sustainability thereof, the composition I is substantially free of an ionic surfactant such as an anionic surfactant, a cationic surfactant, and an amphoteric surfactant.

In the present description, the term “substantially free of” means that the content of the ionic surfactant in composition I is preferably 5% by mass or less, more preferably 1% by mass or less, further preferably 0.5% by mass or less, still more preferably 0.1% by mass or less.

### (Component (H): Acid)

The composition I preferably further contains (H) acid from the viewpoint of stably forming a polyion complex.

An organic acid or an inorganic acid can be used as component (H), and an organic acid is preferable from the viewpoint of being used in a cosmetic composition.

Examples of the organic acid include carboxylic acid-based compounds and sulfonic acid-based compounds other than component (B), and preferably the compounds having a molecular weight of 500 or less, more preferably 200 or less.

Examples of the carboxylic acid-based compound include: aliphatic monocarboxylic acids with 4 or less carbon atoms, such as acetic acid, propionic acid, and butanoic acid; aromatic monocarboxylic acids such as benzoic acid; aliphatic dicarboxylic acids such as malonic acid, succinic acid, glutaric acid, adipic acid, maleic acid, and fumaric acid; aromatic dicarboxylic acids such as phthalic acid and isophthalic acid; polycarboxylic acids such as polyglutamic acid; hydroxycarboxylic acids such as lactic acid, malic acid, glycolic acid, hydroxyacrylic acid, glyceric acid, tartaric acid, and citric acid; and acidic amino acids such as glutamic acid and aspartic acid, and one or two or more of these can be used.

Examples of the sulfonic acid-based compound include: aliphatic sulfonic acids such as methanesulfonic acid and ethanesulfonic acid; and aromatic sulfonic acids such as p-toluenesulfonic acid and naphthalenesulfonic acid, and one or two or more of these can be used.

From the viewpoint of stably forming a polyion complex to form a highly stable cosmetic composition and the viewpoint of using it in a cosmetic composition, the organic acid is preferably one or more selected from the group consisting of aliphatic dicarboxylic acids, hydroxycarboxylic acids, and aromatic sulfonic acids, more preferably one or more selected from the group consisting of succinic acid, lactic acid, malic acid, glycolic acid, p-toluenesulfonic acid, and naphthalenesulfonic acid, further preferably one or more selected from the group consisting of succinic acid and lactic acid, and still more preferably lactic acid.

In formulation, at least a part of the organic acid may be in the state of an organic acid salt. From the viewpoint of use in cosmetic compositions and the like and the viewpoint of availability, the salts of the organic acids are preferably alkali metal salts of the organic acids or alkali metal salts, more preferably alkali metal salts, further preferably one or more selected from the group consisting of sodium salts and potassium salts, and still more preferably sodium salts.

From the viewpoint of ease of forming the polyion complex and the viewpoint of improving the stability of the composition, the ratio of the organic acid salt in the total amount of the organic acid and the organic acid salt is preferably 50% by mass or less, more preferably 20% by mass or less, further preferably 5% by mass or less, and may be 0% by mass. The ratio (% by mass) of the organic acid salt herein means % by mass converted into an organic acid.

### (Water)

The composition I preferably contains water from the viewpoint of forming an emulsified composition. The water used in the composition I is preferably deionized water or distilled water. Tap water, groundwater, or the like sterilized with hypochlorous acid or the like may be used as long as the stability of the composition is not impaired.

### (Other Components)

The composition I can be blended with, optional components, organic acids or inorganic acids, anionic surfactants, nonionic surfactants, amphoteric surfactants, aromatic alcohols such as phenoxyethanol, non-aromatic polyols such as dipropylene glycol, oils other than components (C), (D), and (F), antioxidants, antidandruffs, vitamins, fungicides, anti-inflammatory agents, preservatives, chelating agents, moisturizers, pearls, ceramides, fragrances, plant extracts, UV absorbers, and pH regulators.

(pH)

From the viewpoint of the stability of the composition, the pH of the composition I is preferably 1.0 or more, more preferably 3.0 or more, preferably 7.5 or less, more preferably 7.0 or less, further preferably 6.0 or less, and still more preferably 5.0 or less, as the pH of an aqueous solution obtained by diluting the composition I with water 20 times at 25 °C.

The pH can be measured by using a pH meter.

### (Content)

From the viewpoint of ease of forming a polyion complex, ease of conditioning the shape of the object to be treated and improvement of the sustainability thereof, particularly the viewpoint of the effect of suppressing the spread the hair and the improvement of the sustainability thereof, and the viewpoint of improving the stability of the composition, the content or formulation amount of each component in the composition I is preferably as follows.

From the viewpoint of ease of forming a polyion complex and the viewpoint of the effect of the present invention, the content of component (A) in the composition I is preferably 0.01% by mass or more, more preferably 0.02% by mass or more, further preferably 0.05% by mass or more, still more preferably 0.1% by mass or more, and still more preferably 0.2% by mass or more. In addition, from the viewpoint of improving the stability of the composition, the content of component (A) in the composition I is preferably 3.0% by mass or less, more preferably 2.5% by mass or less, further preferably 2.0% by mass or less, still more preferably 1.5% by mass or less, and still more preferably 0.5% by mass or less. The content of component (A) in the composition I is preferably 0.01% by mass or more and 3.0% by mass or less, more preferably 0.02% by mass or more and 2.5% by mass or less, further preferably 0.05% by mass or more and 2.0% by mass or less, still more preferably 0.1% by mass or more and 1.5% by mass or less, still more preferably 0.2% by mass or more and 1.5% by mass or less, and still more preferably 0.2% by mass or more and 0.5% by mass or less.

From the viewpoint of ease of forming a polyion complex, the viewpoint of improving the film forming ability, and the improvement of the stability of the composition, the content of component (B) in the composition I is preferably 0.005% by mass or more, more preferably 0.01% by mass or more, and further preferably 0.02% by mass or more. In addition, from the viewpoint of improving the stability of the composition, the content of component (B) in the composition I is preferably 3.0% by mass or less, more preferably 2.0% by mass or less, further preferably 1.5% by mass or less, still more preferably 1.0% by mass or less, still more preferably 0.5% by mass or less, still more preferably 0.3% by mass or less, still more preferably 0.2% by mass or less, still more preferably 0.1% by mass or less, and still more preferably 0.08% by mass or less. The content of component (B) in the composition I is preferably 0.005% by mass or more and 3.0% by mass or less, more preferably 0.01% by mass or more and 2.0% by mass or less, further preferably 0.02% by mass or more and 1.5% by mass or less, still more preferably 0.02% by mass or more and 1.0% by mass or less, still more preferably 0.02% by mass or more and 0.5% by mass or less, still more preferably 0.02% by mass or more and 0.3% by mass or less, still more preferably 0.02% by mass or more and 0.2% by mass or less, still more preferably 0.02% by mass or more and 0.1% by mass or less, and still more preferably 0.02% by mass or more and 0.08% by mass or less.

From the viewpoint of ease of conditioning the shape of the object to be treated and the improvement of the sustainability thereof, particularly, the viewpoint of the effect of suppressing the spread of the hair and the improvement of the sustainability thereof, the total content of component (A) and component (B) in the composition I is preferably 0.015% by mass, more preferably 0.02% by mass or more, further preferably 0.03% by mass or more, still more preferably 0.04% by mass or more, further preferably 0.05% by mass or more, still more preferably 0.07% by mass or more, still more preferably 0.1% by mass or more, still more preferably 0.12% by mass or more, still more preferably 0.2% by mass or more, and still more preferably 0.22% by mass or more. In addition, from the viewpoint of improving the stability of the composition, the total content of component (A) and component (B) in the composition I is preferably 6.0% by mass or less, more preferably 5.0% by mass or less, further preferably 4.0% by mass or less, still more preferably 3.8% by mass or less, still more preferably 3.5% by mass or less, still more preferably 3.0% by mass or less, still more preferably 2.5% by mass or less, still more preferably 2.0% by mass or less, still more preferably 0.8% by mass or less, still more preferably 0.7% by mass or less, still more preferably 0.6% by mass or less, and still more preferably 0.58% by mass or less. The total content of component (A) and component (B) of the composition I is preferably 0.015% by mass or more and 6.0% by mass or less, more preferably 0.02% by mass or more and 5.0% by mass or less, further preferably 0.03% by mass or more and 4.5% by mass or less, still more preferably 0.04% by mass or more and 4.0% by mass or less, still more preferably 0.05% by mass or more and 3.8% by mass or less, still more preferably 0.07% by mass or more and 3.5% by mass or less, still more preferably 0.07% by mass or more and 3.5% by mass or less, still more preferably 0.1% by mass or more and 3.0% by mass or less, still more preferably 0.12% by mass or more and 2.5% by mass or less, still more preferably 0.22% by mass or more and 2.0% by mass or less, still more preferably 0.22% by mass or more and 0.8% by mass or less, still more preferably 0.22% by mass or more and 0.7% by mass or less, still more preferably 0.22% by mass or more and 0.6% by mass or less, and still more preferably 0.22% by mass or more and 0.58% by mass or less.

From the viewpoint of ease of forming a polyion complex and the viewpoint of the effect of the present invention, the ratio of the content of component (A) and component (B) contained in the composition I, the mass ratio [(A)/(B)] of component (A) to component (B), is preferably 1.0 or more, more preferably 2.0 or more, further preferably 3.0 or more, still more preferably 4.0 or more, and still more preferably 5.0 or more, and in addition from the viewpoint of improving the stability of the composition, preferably 20 or less, more preferably 15 or less, further preferably 12 or less, and still more preferably 10 or less. The mass ratio [(A)/(B)] is preferably 1.0 or more and 20 or less, more preferably 2.0 or more and 15 or less, further preferably 3.0 or more and 12 or less, still more preferably 4.0 or more and 12 or less, and still more preferably 5.0 or more and 10 or less.

In the composition of the present invention, the mass ratio of component (A) to component (B) works more predominantly than the ratio of the cation charge to anion charge of component (A) and component (B) for the effect of the present invention.

From the viewpoints of ease of forming a polyion complex and the interaction with the film formed by composition II, the ratio of the number of moles of the cation charge of component (A) to the number of moles of the anion charge of component (B) in the composition I is preferably 10/90 to 95/5, more preferably 20/80 to 95/5, and further preferably 25/75 to 90/10.

When the composition I contains (G) a nonionic polymer, from the viewpoint of ease of conditioning the shape of the object to be treated and the improvement of the sustainability thereof, particularly the viewpoint of the effect of suppressing the spread of the hair and the improvement of the sustainability thereof, and the viewpoint of suitably exhibiting the improvement of the stability of the composition, the content of component (G) in the composition I is preferably 0.01% by mass or more, more preferably 0.02% by mass or more, further preferably 0.05% by mass or more, still more preferably 0.1% by mass or more, and still more preferably 0.2% by mass or more. In addition, from the viewpoint of improving the stability of the composition, the content of component (G) in the composition I is preferably 3.0% by mass or less, more preferably 2.5% by mass or less, further preferably 2.0% by mass or less, still more preferably 1.5% by mass or less, still more preferably 1.0% by mass or less, and still more preferably 0.6% by mass or less. The content of component (G) in the composition I is preferably 0.01% by mass or more and 3.0% by mass or less, more preferably 0.02% by mass or more and 2.5% by mass or less, further preferably 0.05% by mass or more and 2.0% by mass or less, still more preferably 0.1% by mass or more and 1.5% by mass or less, still more preferably 0.1% by mass or more and 1.0% by mass or less, and still more preferably 0.2% by mass or more and 0.6% by mass or less.

When the composition I contains (H) an acid, from the viewpoint of ease of forming a polyion complex, the content of component (H) in the composition I is preferably 0.01% by mass or more, more preferably 0.02% by mass or more, further preferably 0.03% by mass or more, still more preferably 0.05% by mass or more, still more preferably 0.1% by mass or more, and still more preferably 0.2% by mass or more. In addition, from the viewpoint of ease of forming of a polyion complex and the viewpoint of improving the stability of the composition, the content of component (H) in the composition I is preferably 5.0% by mass or less, more preferably 3.0% by mass or less, further preferably 2.5% by mass or less, and further preferably 0.5% by mass or less. The content of component (H) in the composition I is preferably 0.01% by mass or more and 5.0% by mass or less, more preferably 0.02% by mass or more and 3.0% by mass or less, further preferably 0.03% by mass or more and 3.0% by mass or less, still more preferably 0.05% by mass or more and 3.0% by mass or less, still more preferably 0.05% by mass or more and 3.0% by mass or less, still more preferably 0.1% by mass or more and 3.0% by mass or less, still more preferably 0.1% by mass or more and 2.5% by mass or less, still more preferably 0.2% by mass or more and 2.5% by mass or less, and still more preferably 0.2% by mass or more and 0.5% by mass or less.

When the organic acid is formulated in the state of an organic acid salt, the above content is an amount converted into an organic acid.

When the composition I contains water, from the viewpoint of ease of forming a polyion complex and the viewpoint of improving the stability of the composition, the water content of the composition I is preferably 10% by mass or more, more preferably 20% by mass or more, and further preferably 30% by mass or more. In addition, from the viewpoint of improving the stability of the composition and the viewpoint of the degree of freedom of formulation, the water content of the composition I is preferably 99% by mass or less, more preferably 95% by mass or less, and still more preferably 90% by mass or less. The content of water in the composition I is preferably 10% by mass or more and 99% by mass or less, more preferably 20% by mass or more and 95% by mass or less, and further preferably 30% by mass or more and 90% by mass or less. The content of water in the composition may be the balance of the component (A) and component (B), and optionally contained component (G) and component (H).

### (Viscosity)

From the viewpoint of ease of forming a polyion complex, ease of conditioning the shape of the object to be treated, and improvement of the sustainability thereof, particularly from the viewpoint of the effect of suppressing the spread of the hair and improving the sustainability thereof, and from the viewpoint of improving the stability of the composition, the viscosity of the composition I at 30 °C is 1 mPa-s or more, preferably 5 mPa-s or more, more preferably 10 mPa-s or more. From the viewpoint of ease of forming a polyion complex and handling, the viscosity of the composition I is preferably 10,000 mPa-s or less, more preferably 5,000 mPa-s or less, further preferably 1,500 mPa-s or less. The viscosity of the composition I at 30 °C is preferably 1 mPa-s or more and 10,000 mPa-s or less, more preferably 5 mPa-s or more and 5,000 mPa-s or less, and further preferably 10 mPa-s or more and 1,500 mPa-s or less.

The viscosity of the composition I at 30 °C can be measured by the method specifically described in Examples.

### (Method of Producing Composition I)

The method of producing composition I is not particularly limited, and each of the components can be dissolved or dispersed in water for production. From the viewpoint of ease of conditioning the shape of the object to be treated and the improvement of the sustainability thereof, particularly the viewpoint of the effect of suppressing the spread of the hair and the improvement of the sustainability thereof, and the viewpoint of being capable of producing a more stable composition, composition I is preferably produced by a method having the following steps M1 to M3 in order.
Step M1: a step of dissolving or dispersing (B) an anionic polymer in water to prepare a solution 1 containing (B) the anionic polymer.
Step M2: a step of mixing the solution 1 with (H) an acid or an aqueous solution thereof to prepare a mixture 2 containing (B) the anionic polymer, (H) the acid, and water.
Step M3: a step of mixing the mixture 2 and a solution obtained by dissolving or dispersing (A) a cationic polymer in water to prepare a mixture 3 containing (A) the cationic polymer, (B) the anionic polymer, (H) the acid, and water.

### (Application to Hair)

When step 1 is performed first and then step 2 is performed, composition I is applied directly to the hair. In this case, the film formed by composition I has good adhesion to the hair as a base coat.

When the step 1 is performed after the step 2, composition I is applied to the hair to which composition II has been applied first. In this case, the film formed by composition I tends to achieve an excellent texture as a topcoat regardless of the diameter of the hair and the degree of damage.

The method of applying the composition I to the hair may be any method as long as the composition I can be brought into contact with the hair, and examples thereof include a method of applying the composition to dry or wet hair and a method of immersing dry or wet hair in the composition.

From the viewpoint of ease of conditioning the shape of the object to be treated and the improvement of the sustainability thereof, particularly, the viewpoint of the effect of suppressing the spread of the hair and the improvement of the sustainability thereof, and the balance of economic efficiency, the amount of composition I applied to the hair is, in dry mass of hair: mass of composition I, preferably 1:0.01 to 1:5, more preferably 1:0.05 to 1:1, and still more preferably 1:0.1 to 1:0.5.

### {Step 2}

Step 2: a step of applying composition II including the following component (C) and component (D) to hair.

Composition II is a hair cosmetic composition for direct application to hair. Specific product forms thereof include hair shampoos, hair rinses, hair conditioners, hair treatments (including non-rinsing types), hair styling agents, hair colors, and permanent care products. Among these, from the viewpoint of effectiveness of the effect of the invention, hair treatments or hair styling agents are preferable.

The form of composition II is preferably an emulsified composition, and more preferably an oil-in-water emulsified composition from the viewpoint of improving the stability of the composition and improving the feel of use.

Hereinafter, each component contained in composition II is explained.

### (Component (C): Modified Silicone)

Component (C) is one or more modified silicones selected from the group consisting of polyglycerin-modified silicones and amino-modified silicones. Comprising component (C) in the composition II is assumed to exhibit the viewpoint of interaction with the base coat, the effect of improving the texture, and the effect of sustaining the effect, particularly the effect of improving the texture of the hair. In addition, hydrogen bonds can be formed between component (B) and component (C), and therefore it is assumed that a network with high strength is formed.

### Polyglycerin-Modified Silicone

Polyglycerin-modified silicone is a silicone having a polyglycerin chain in the molecule. The position of introduction of the polyglycerin chain is optional and the form of introduction may be any of one-ended type, two-ended type, and side chain type, and the silicone preferably has a polyglycerin chain at the side chain or the end of the silicone chain, more preferably has a monovalent polyglyceryl group at the side chain or the end of the silicone chain.

Specific examples of polyglycerin-modified silicone include polyglyceryl-3-polydimethylsiloxyethyl dimethicone, polyglyceryl-3-disiloxane dimethicone, lauryl polyglyceryl-3-polydimethylsiloxyethyl dimethicone, cetyldiglyceryltris (trimethylsiloxy)silylethyldimethicone, and bis(polyglyceryl-3-oxyphenylpropyl)dimethicone.

Examples of commercially available products of polyglycerin-modified silicone include: “KF-6100”, “KF-6104”, “KF6106”, “KF-6105” manufactured by Shin-Etsu Chemical Co., Ltd.; “DOWSI ES-5600 Silicone Glycerol Emulsifier” manufactured by Toray Dow Co., Ltd.; and “SOFCARE GS-G” manufactured by Kao Corporation.

### Amino-Modified Silicone

Examples of amino-modified silicone include silicone having an amino group- or ammonium group-containing group in the main chain or side chain and having no polyether structure, such as dimethylpolysiloxane (dimethicone) and methylphenylpolysiloxane, and preferably silicone represented by the following general formula (2).

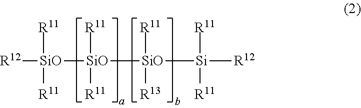

wherein R¹¹ each independently represents a methyl group or a phenyl group, and R¹² each independently represents an alkyl group having 1 or more and 30 or less carbon atoms, a hydroxy group, or R¹³; R¹³ represents a monovalent group represented by —R¹⁴—Z¹ (R¹⁴ represents a single bond or a divalent hydrocarbon group having 1 or more and 20 or less carbon atoms, and Z¹ represents a primary to tertiary amino group-containing group or an ammonium group-containing group); and a is a number of 0 or more and 3000 or less, b is a number of 1 or more and 3000 or less, and a+b is a number of 3,000 or more and 20,000 or less.

In the general formula (2), R¹¹ is preferably a methyl group, and R¹² is preferably a methyl group or R¹³.

R¹⁵ is a monovalent group represented by —R¹⁴—Z¹, and R¹⁴ is preferably a divalent hydrocarbon group having 1 or more and 20 or less carbon atoms, more preferably an alkylene group having 1 or more and 20 or less carbon atoms more preferably a linear or branched alkylene group having 1 or more and 6 or less carbon atoms, still more preferably a methylene group, an ethylene group, a trimethylene group, a propylene group, a tetramethylene group, or a hexamethylene group, and still more preferably a trimethylene group or a propylene group.

Z¹ is a primary to tertiary amino group-containing group or an ammonium group-containing group, preferably an amino group-containing group represented by —N(R¹⁵)₂, —NR¹⁵(CH₂)_{c}N(R¹⁶)₂, or —NR¹⁵(CH₂)_{c}N(R¹⁶)CO—R¹⁷, or an ammonium group-containing group represented by —N⁺(R¹⁵)₃A⁻ and —NR¹⁵(CH₂)_{c}N⁺(R¹⁶)₃A⁻. Herein, R¹⁵ and R¹⁶ each independently represent a hydrogen atom or an alkyl group having 1 or more and 3 or less carbon atoms, and are preferably a hydrogen atom or a methyl group. R¹⁷ represents an alkyl group having 1 or more and 3 or less carbon atoms. A is a counter anion and represents Cl, Br or I. c represents a number of 1 or more and 6 or less, preferably 2 or more and 4 or less.

In the general formula (2), R¹³ is preferably —(CH₂)₃—NH₂, —(CH₂)₃—N(CH₃)₂, —(CH₂)₃—NH—(CH₂)₂—NH₂, or —(CH₂)₂—NH—(CH₂)₂—N(CH₃)₂, and more preferably —(CH₂)₃—NH₂ or —(CH₂)₃—NH—(CH₂)₂—NH₂.

Preferable silicones represented by the general formula (2) include one or more selected from the group consisting of aminoethylaminopropyldimethicone [amodimeticone], aminopropyldimethicone, bis(aminopropyl)dimethicone, and bis(cetearyl)amodimethicone.

Examples of commercially available products of amino-modified silicone include “Silicone SF 8457 C” and “DOWSIL 2-8566 AMINO FLUID” manufactured by Dow Toray Co., Ltd. In addition, “DOWSIL CF 1046” manufactured by Dow Toray Co., Ltd., which is a mixture of amino-modified silicone and (D) dimethylpolysiloxane, which will be described later, can be used.

Component (C) may be used singly or in combination of two or more. Among the above, amino-modified silicone is preferable, from the viewpoint of interaction with the base coat, the effect of improving the texture and the sustainability of the effect, and the viewpoint of providing a smooth and moist feeling to the object to be treated.

### (Component (D): Dimethylpolysiloxane)

Component (D) is dimethylpolysiloxane. Comprising component (C) and component (D) in the composition II is assumed to achieve the effect of improving texture and the sustainability of the effect and to exhibit the effect of providing lightness and naturalness to the object to be treated.

The dimethylpolysiloxane used as component (D) is preferably low in volatility, and from this viewpoint, the viscosity at 25 °C is preferably 500 mm²/s or more, more preferably 700 mm²/s or more, further preferably 1,000 mm²/s or more.

From the effect of providing lightness and naturalness to the object to be treated, component (D) more preferably contains (D1) dimethylpolysiloxane having a viscosity at 25 °C of 1,000 mm²/s or more and 10,000 mm²/s or less and (D2) dimethylpolysiloxane having a viscosity at 25 °C of 300,000 mm²/s or more and 5,000,000 mm²/s or less.

The viscosity of component (D) is a value measured at 25 °C in accordance with the “liquid viscosity measuring method” specified in JIS Z8803:2011 or ASTM D 445-46T, and, for example, can be measured by using a Ubbelohde viscometer.

When component (D) contains component (D1) and component (D2), from the effect of improving the texture and the effect of sustaining the effect, the mass ratio [(D1)/(D2)] of component (D1) to component (D2) is preferably 0.5 or more, more preferably 1.0 or more, further preferably 2.0 or more, and is preferably 20 or less, more preferably 10 or less, further preferably 5.0 or less. The mass ratio [(D1)/(D2)] is preferably 0.5 or more and 20 or less, more preferably 1.0 or more and 10 or less, and further preferably 2.0 or more and 5.0 or less.

When component (D) contains component (D1) and component (D2), from the effect of providing lightness and naturalness to the object to be treated, the total content of component (D1) and component (D2) in component (D) is preferably 50% by mass or more, more preferably 60% by mass or more, further preferably 70% by mass or more, still more preferably 80% by mass or more, still more preferably 90% by mass or more, and 100% by mass or less.

### (Component (E): Cationic Surfactant)

The composition II preferably further contains (E) a cationic surfactant, from the viewpoint of suitably exhibiting the effect of improving the texture and the effect of sustaining the effect.

Examples of the cationic surfactant include (i) alkyltrimethylammonium salt, (ii) alkoxyalkyltrimethylammonium salt, (iii) dialkyldimethylammonium salt, (iv) alkylamide alkyltrimethylammonium salt, (v) alkyldimethylamine and a salt thereof, (vi) alkoxyalkyldimethylamine and a salt thereof, and (vii) alkylamide alkyldimethylamine and a salt thereof.

Examples of (I) the alkyltrimethylammonium salt include an alkyltrimethylammonium salt having an alkyl group preferably having 12 or more and 24 or less carbon atoms, more preferably 16 or more and 24 or less carbon atoms, and specific examples thereof include cetyltrimethylammonium chloride (cetrimonium chloride), stearyltrimethylammonium chloride (stearyltrimonium chloride), and behenyltrimethylammonium chloride (behentrimonium chloride).

Examples of (ii) the alkoxyalkyltrimethylammonium salt include an alkoxyalkyltrimethylammonium salt having an alkoxy group preferably having 12 or more and 22 or less carbon atoms, and more preferably 16 or more and 20 or less carbon atoms, and specific examples thereof include stearoxypropyltrimethylammonium chloride, stearoxyethyltrimethylammonium chloride, and stearoxyhydroxypropyltrimethylammonium chloride.

Examples of the (iii) dialkyldimethylammonium salt include dialkyldimethylammonium salts having an alkyl group preferably having 12 or more and 22 or less carbon atoms, and more preferably 16 or more and 20 or less carbon atoms, and specific examples thereof include distearyldimethylammonium chloride. Examples of the (iv) alkylamide alkyltrimethylammonium salt include alkylamide alkyltrimethylammonium salts having an alkyl group preferably having 11 or more and 21 or less carbon atoms, and more preferably 13 or more and 19 or less carbon atoms, and specific examples thereof include palmitamidpropyltrimethylammonium chloride (palmitamidepropyltrimonium chloride).

(v) Alkyldimethylamine, (vi) alkoxyalkyldimethylamine, and (vii) alkylamide alkyldimethylamine react with an acid to form a tertiary amine salt and become a cationic surfactant.

The alkyl group in (v) alkyldimethylamine and a salt thereof, and (vi) alkoxyalkyldimethylamine and a salt thereof is an alkyl group preferably having 12 or more and 22 or less carbon atoms, and more preferably 16 or more and 20 or less carbon atoms.

The alkyl group in (vii) alkylamide alkyldimethylamine and a salt thereof is an alkyl group preferably having 11 or more and 21 or less carbon atoms, and more preferably 15 or more and 19 or less carbon atoms.

The amines (v) to (vii) may be previously reacted with an acid and formulated as a salt in composition II, or may be formulated as amines in composition II and the acid may be further formulated in composition II to form a salt in the composition. Therefore, the amine and a salt thereof are defined herein as a cationic surfactant. In addition, the content or the formulation amount is converted by the mass of the amine.

Examples of the amine salts of (v) to (vii) include salts with organic acids or inorganic acids. Examples of the organic acid include monocarboxylic acids such as acetic acid and propionic acid; dicarboxylic acids such as malonic acid, succinic acid, glutaric acid, adipic acid, maleic acid, fumaric acid, and phthalic acid; polycarboxylic acids such as polyglutamic acid; hydroxycarboxylic acids such as glycolic acid, lactic acid, hydroxyacrylic acid, glyceric acid, malic acid, tartaric acid, and citric acid; and acidic amino acids such as glutamic acid and aspartic acid. Examples of the inorganic acid include hydrochloric acid, sulfuric acid, and phosphoric acid. Among these, an organic acid is preferable, and one or more selected from the group consisting of a dicarboxylic acid, a hydroxycarboxylic acid, and an acidic amino acid is more preferable. The dicarboxylic acid is more preferably one or more selected from the group consisting of maleic acid and succinic acid. The hydroxycarboxylic acid is more preferably one or more selected from the group consisting of glycolic acid, lactic acid, and malic acid. The acidic amino acid is more preferably glutamic acid.

Examples of (v) alkyldimethylamine and a salt thereof include N,N-dimethylbehenylamine, N,N-dimethylstearylamine, and organic acid salts thereof, and a lactate of N,N-dimethylbehenylamine, glycolate of N,N-dimethylstearylamine, and the like are preferable.

Examples of (vi) alkoxyalkyldimethylamine and a salt thereof include N,N-dimethyl-3-hexadecyloxypropylamine, N,N-dimethyl-3-octadecyloxypropylamine, and organic acid salts thereof, and N,N-dimethyl-3-hexadecyloxypropylamine or a lactate thereof and N,N-dimethyl-3-octadecyloxypropylamine (stearoxypropyldimethylamine) or a lactate thereof are preferable.

Examples of (vii) alkylamide alkyldimethylamine and a salt thereof include N[3-(dimethylamino)propyl]docosanamide, N-[3-(dimethylamino)propyl]stearamide, and organic acid salts thereof, and N-[3-(dimethylamino)propyl]docosanamide lactate and N-[3-(dimethylamino)propyl]stearamide glycolate are preferable.

Component (E) may be used singly or in combination of two or more.

Among the above, from the viewpoint of improving the stability of the composition, component (E) is: preferably one or more selected from the group consisting of (i) alkyltrimethylammonium salt, (ii) alkoxyalkyltrimethylammonium salt, (iii) dialkyldimethylammonium salt, (iv) alkylamide alkyltrimethylammonium salt, (v) alkyldimethylamine and a salt thereof, (vi) alkoxyalkyldimethylamine and a salt thereof, and (vii) alkylamide alkyldimethylamine and a salt thereof; more preferably one or more selected from the group consisting of (i) alkyltrimethylammonium salt and (vi) alkoxyalkyldimethylamine and a salt thereof; more preferably one or more selected from the group consisting of cetyltrimethylammonium chloride, stearyltrimethylammonium chloride, behenyltrimethylammonium chloride, N,N-dimethyl-3-hexadecyloxypropylamine or a lactate thereof, and N,N-dimethyl-3-octadecyloxypropylamine (stearoxypropyldimethylamine) or a lactate thereof; still more preferably one or more selected from the group consisting of behenyltrimethylammonium chloride and N,N-dimethyl-3-octadecyloxypropylamine (stearoxypropyldimethylamine) or a lactate thereof.

### (Component (F): Higher Alcohol)

From the viewpoint of improving the stability of the composition, the composition II preferably further contains (F) higher alcohol.

Examples of the higher alcohol include aliphatic monohydric alcohols having 12 or more carbon atoms, preferably 12 or more and 22 or less carbon atoms. Examples of the higher alcohol include one or more selected from the group consisting of lauryl alcohol, cetyl alcohol, myristyl alcohol, oleyl alcohol, stearyl alcohol, isostearyl alcohol, 2-octyldodecanol, behenyl alcohol, and cetostearyl alcohol.

Among the above, from the viewpoint of further improving the feel of the object to be treated, the higher alcohol is preferably one or more selected from the group consisting of cetyl alcohol, myristyl alcohol, stearyl alcohol, and behenyl alcohol.

### (Water)

The composition II preferably contains water from the viewpoint of forming an emulsified composition. The water used in the composition II is preferably deionized water or distilled water. Tap water, groundwater, or the like sterilized with hypochlorous acid or the like may be used as long as the stability of the composition is not impaired.

### (Other Components)

The composition II can be blended with, optional components, organic acids or inorganic acids, anionic surfactants, nonionic surfactants, amphoteric surfactants, aromatic alcohols such as phenoxyethanol, non-aromatic polyols such as dipropylene glycol, oils other than components (C), (D), and (F), antioxidants, antidandruffs, vitamins, fungicides, anti-inflammatory agents, preservatives, chelating agents, moisturizers, pearls, ceramides, fragrances, plant extracts, UV absorbers, and pH regulators.

(pH)

From the viewpoint of adsorption to hair and stability of the composition, the pH of the above composition II is, as the pH of an aqueous solution of composition II diluted 20-fold with water at 25 °C, preferably 3.0 or more, and more preferably 3.2 or more. In addition, from the viewpoint of improving the stability of the composition, the pH is preferably 7.5 or less, more preferably 7.0 or less, further preferably 6.0 or less, and still more preferably 5.0 or less.

The pH can be measured by using a pH meter.

### (Viscosity)

From the viewpoint of ease of forming a polyion complex, ease of conditioning the shape of the object to be treated, and improvement of the sustainability thereof, particularly from the viewpoint of the effect of suppressing the spread of the hair and improving the sustainability thereof, and from the viewpoint of improving the stability of the composition, the viscosity of the composition II at 30 °C is 10,000 mPa-s or more, preferably 30,000 mPa-s or more, more preferably 60,000 mPa-s or more. From the viewpoint of ease of forming a polyion complex and handling, the viscosity of the composition II is preferably 200,000 mPa-s or less, more preferably 150,000 mPa-s or less, further preferably 120,000 mPa-s or less. The viscosity of the composition I at 30 °C is preferably 10,000 mPa-s or more and 200,000 mPa-s or less, more preferably 30,000 mPa-s or more and 150,000 mPa-s or less, and further preferably 60,000 mPa-s or more and 120,000 mPa-s or less.

The viscosity of the composition II at 30 °C can be measured by the method specifically described in Examples.

### (Content)

From the viewpoint of the effect of improving the texture and achieving the sustainability of the effect, and providing a smooth and moist feeling to the object to be treated, the content of component (C) in the composition II is preferably 0.01% by mass or more, more preferably 0.02% by mass or more, further preferably 0.05% by mass or more, still more preferably 0.1% by mass or more, and still more preferably 0.5% by mass or more. In addition, from the viewpoint of improving the stability of the composition, the content of component (C) in the composition II is preferably 5.0% by mass or less, more preferably 3.0% by mass or less, further preferably 2.0% by mass or less, and still more preferably 1.0% by mass or less. The content of component (C) in the composition II is preferably 0.01% by mass or more and 5.0% by mass or less, more preferably 0.02% by mass or more and 3.0% by mass or less, further preferably 0.05% by mass or more and 2.0% by mass or less, still more preferably 0.1% by mass or more and 1.0% by mass or less, and still more preferably 0.5% by mass or more and 1.0% by mass or less.

From the viewpoint of the effect of improving the texture and achieving the sustainability of the effect, and providing a lightness and naturalness to the object to be treated, the content of component (D) in the composition II is preferably 0.05% by mass or more, more preferably 0.1% by mass or more, further preferably 0.3% by mass or more, still more preferably 0.5% by mass or more, and still more preferably 1.0% by mass or more. In addition, from the viewpoint of improving the stability of the composition, the content of component (D) in the composition II is preferably 15% by mass or less, more preferably 10% by mass or less, further preferably 8.0% by mass or less, still more preferably 5.0% by mass or less, and still more preferably 4.0% by mass or less. The content of component (D) in the composition II is preferably 0.05% by mass or more and 15% by mass or less, more preferably 0.1% by mass or more and 10% by mass or less, and further preferably 0.3% by mass or more and 8.0% by mass or less, still more preferably 0.5% by mass or more and 5.0% by mass or less, and still more preferably 1.0% by mass or more and 4.0% by mass or less.

From the viewpoint of the effect of improving the texture and sustainability of the effect, the total content of component (C) and component (D) in the composition II is preferably 0.06% by mass or more, more preferably 0.1% by mass or more, further preferably 0.3% by mass or more, still more preferably 0.5% by mass or more, still more preferably 0.6% by mass or more, and still more preferably 1.0% by mass or more. In addition, from the viewpoint of improving the stability of the composition, the total content of component (C) and component (D) in the composition II is preferably 20% by mass or less, more preferably 15% by mass or less, further preferably 10% by mass or less, still more preferably 6.0% by mass or less, and still more preferably 5.0% by mass or less. The total content of component (C) and component (D) in the composition II is preferably 0.06% by mass or more and 20% by mass or less, more preferably 0.1% by mass or more and 15% by mass or less, further preferably 0.3% by mass or more and 10% by mass or less, still more preferably 0.5% by mass or more and 10% by mass or less, and still more preferably 0.6% by mass or more and 6.0% by mass or less, and still more preferably 1.0% by mass or more and 5.0% by mass or less.

From the viewpoint of the effect of improving the texture and sustainability of the effect, the ratio of the contents of component (C) and component (D) in the composition II, the mass ratio [(C)/(D)] of component (C) to component (D) is preferably 0.02 or more, more preferably 0.05 or more, further preferably 0.1 or more, further preferably 0.2 or more, and preferably 20 or less, more preferably 10 or less, further preferably 5.0 or less, still more preferably 2.0 or less, still more preferably 1.0 or less, still more preferably 0.5 or less still more preferably 0.3 or less. The mass ratio [(C)/(D)] is preferably 0.02 or more and 20 or less, more preferably 0.02 or more and 10 or less, further preferably 0.05 or more and 5.0 or less, still more preferably 0.05 or more and 2.0 or less, still more preferably 0.1 or more and 1.0 or less, still more preferably 0.1 or more and 0.5 or less, and still more preferably 0.2 or more and 0.3 or less.

When the composition II contains component (E), from the viewpoint of the stability of the composition, the content of component (E) in the composition II is preferably 0.1% by mass or more, more preferably 0.2% by mass or more, further preferably 0.5% by mass or more, and further preferably 2.0% by mass or more. In addition, from the viewpoint of improving the stability of the composition, the content of component (E) in the composition II is preferably 10% by mass or less, more preferably 5.0% by mass or less, further preferably 3.5% by mass or less, and further preferably 3.0% by mass or less. The content of component (E) in the composition II is preferably 0.1% by mass or more and 10% by mass or less, more preferably 0.2% by mass or more and 5.0% by mass or less, further preferably 0.5% by mass or more and 3.5% by mass or less, and still more preferably 2.0% by mass or more and 3.0% by mass or less.

When the composition II contains the component (F), from the viewpoint of the stability of the composition, the content of component (F) in the composition II is preferably 0.1% by mass or more, more preferably 0.2% by mass or more, further preferably 0.5% by mass or more, still more preferably 1.0% by mass or more, and still more preferably 5.0% by mass or more. In addition, from the viewpoint of improving the stability of the composition, the content of component (F) in the composition II is preferably 15% by mass or less, more preferably 12% by mass or less, and further preferably 10% by mass or less. The content of component (F) in the composition II is preferably 0.1% by mass or more and 15% by mass or less, more preferably 0.2% by mass or more and 12% by mass or less, further preferably 0.5% by mass or more and 12% by mass or less, still more preferably 1.0% by mass or more and 10% by mass or less, and still more preferably 5.0% by mass or more and 10% by mass or less.

When the composition II contains water, from the viewpoint of improving the stability of the composition, the water content of the composition II is preferably 10% by mass or more, more preferably 20% by mass or more, and further preferably 30% by mass or more. In addition, from the viewpoint of improving the stability of the composition and the viewpoint of the degree of freedom of formulation, the water content of the composition II is preferably 99% by mass or less, more preferably 95% by mass or less, and still more preferably 90% by mass or less. The water content of the composition II is preferably 10% by mass or more and 99% by mass or less, more preferably 20% by mass or more and 95% by mass or less, and further preferably 30% by mass or more and 90% by mass or less. The water content in the composition may be the balances of the component (C) and component (D), and the component (E) and component (F) that can be optionally contained.

### (Method of Producing Composition II)

The method of producing composition II is not particularly limited, and each of the components can be dissolved or dispersed in water for production. From the viewpoint of ease of conditioning the shape of the object to be treated and improvement of the sustainability, particularly the viewpoint of the effect of suppressing the spread of the hair and improving the sustainability, and the viewpoint of being capable of producing a more stable composition, composition I is preferably produced by a method having the following steps M4 to M5 in order.
Step M4: a step of mixing an aqueous phase (4-1) containing water with an oil phase (4-2) containing (D) a cationic surfactant and (E) a higher alcohol to prepare an emulsion (4)
Step M5: a step of mixing the emulsion (4) with the oil phase (5) containing (C) one or more modified silicones selected from the group consisting of polyglycerin-modified silicones and amino-modified silicones, and (D) dimethylpolysiloxane

### (Application to Hair)

When step 1 is performed first and then step 2 is performed, composition I is applied directly to the hair. In this case, the film formed by composition I has good adhesion to the hair as a base coat.

When the step 1 is performed after the step 2, composition I is applied to the hair to which composition II has been applied first. In this case, the film formed by composition I tends to achieve an excellent texture as a topcoat regardless of the diameter of the hair and the degree of damage.

The method of applying the composition II to the hair may be any method as long as the composition II can be brought into contact with the hair, and examples thereof include a method of applying the composition to the hair and a method of immersing the hair in a dry state or a wet state in the composition.

From the viewpoint of the effect of improving the texture, the sustainability of the effect, and the balance of economic efficiency, the amount of composition II applied to hair is, in dry mass of hair: the mass of composition II, preferably 1:0.01 to 1:5, more preferably 1:0.05 to 1:1, and further preferably 1:0.1 to 1:0.5.

From the viewpoint of improving the lightness and moistness of the hair after application to the hair, the ratio between the amount of composition I applied to the hair and the amount of composition II applied to the hair is, in the mass of composition I: the mass of composition II, preferably 5:1 to 1:5, and more preferably 3:1 to 1:3.

For the embodiments described above, the present invention further discloses the following.

<1>

A method of treating hair, comprising the following step 1 and step 2 in any order and not comprising rinsing the hair with water between step 1 and step 2:
Step 1: a step of applying composition I comprising the following component (A) and component (B) to the hair:
Component (A): a cationic polymer having a viscosity of 1,000 mPa-s or more at 30 °C in a 1% by mass aqueous solution;
Component (B): an anionic polymer; and
Step 2: a step of applying composition II comprising the following component (C) and component (D) to the hair:
Component (C): one or more modified silicones selected from the group consisting of polyglycerin-modified silicones and amino-modified silicones;
Component (D): a dimethylpolysiloxane.
   <2>

The method of treating hair according to <1>, wherein he viscosity of a 1% by mass aqueous solution of component (A) at 30 °C is 1,000 mPa-s or more and 100,000 mPa-s or less, preferably 1,300 mPa-s or more and 80,000 mPa-s or less, more preferably 1,500 mPa-s or more and 50,000 mPa-s or less, further preferably 1,700 mPa-s or more and 30,000 mPa-s or less, and still more preferably 1,800 mPa-s or more and 25,000 mPa-s or less.

<3>

The method of treating hair according to <1> or <2>, wherein the weight average molecular weight (Mw) of component (A) is 5,000 or more and 3,000,000 or less, preferably 10,000 or more and 2,000,000 or less, more preferably 30,000 or more and 2,000,000 or less, further preferably 50,000 or more and 1,500,000 or less, and still more preferably 100,000 or more and 1,500,000 or less.

<4>

The method of treating hair according to any one of <1> to <3>, wherein the cation charge density of component (A) is 0.1 mmol/g or more and 20 mmol/g or less, preferably 0.2 mmol/g or more and 15 mmol/g or less, more preferably 0.2 mmol/g or more and 12 mmol/g or less, further preferably 0.3 mmol/g or more and 10 mmol/g or less, still more preferably 0.3 mmol/g or more and 7.0 mmol/g or less, and still more preferably 0.5 mmol/g or more and 5.0 mmol/g or less.

<5>

The method of treating hair according to any one of <1> to <4>, wherein the component (A) is one or more cationic polymer selected from the group consisting of cationated polyvinyl alcohol, polyethyleneimine, methacryloyl ethyltrimethylammonium salt polymer, quaternized dialkylaminoalkyl (meth)acrylate polymer, diallyl quaternized ammonium salt polymer, methacrylamide propyltrimethylammonium salt polymer, vinylimidazolium trichloride-vinylpyrrolidone copolymer (polyquaternium-16), vinylpyrrolidone-alkylamino (meth)acrylate copolymer, vinylpyrrolidone-alkylamino (meth)acrylate-vinylcaprolactam copolymer, alkylacrylamide-(meth)acrylate-alkylaminoalkylacrylamide-polyethylene glycol (meth)acrylate copolymer, and adipate-dimethylaminohydroxypropylethylenetriamine copolymer.

<6>

The method of treating hair according to any one of <1> to <5>, wherein the component (A) is a polymer containing a constituent unit represented by the following general formula (1):

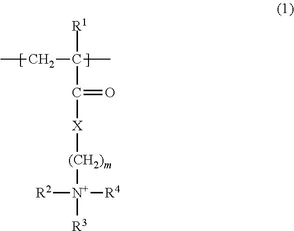

wherein R¹ is a hydrogen atom or a methyl group, R² to R⁴ are each independently an alkyl group having 1 to 4 carbon atoms, X is —O— or —NH—, and m is a number of 1 or more and 4 or less.
<7>

The method of treating hair according to <6>, wherein the constituent unit represented by the general formula (1) include one or more selected from the group consisting of a constituent unit derived from methacryloylethyltrimethylammonium salt, a constituent unit derived from N,N-dimethylaminoethyl methacrylic acid diethyl sulfate, and methacrylamide propyltrimethylammonium salt, and preferably one or more selected from the group consisting of a constituent unit derived from methacryloylethyltrimethylammonium salt and a constituent unit derived from N,N-dimethylaminoethyl methacrylate diethyl sulfate.

<8>

The method of treating hair according to any one of <1> to <7>, wherein component (A) is one or more selected from the group consisting of a methacryloylethyltrimethylammonium salt polymer and a quaternized dialkylaminoalkyl (meth)acrylate polymer; preferably one or more selected from the group consisting of a methacryloyl ethyl trimethylammonium chloride polymer (polyquaternium-37), a methacryloylethyldimethylbetaine-methacryloylethyltrimethylammonium chloridemethoxypolyethylene glycol methacrylate copolymer (polyquaternium-49), a methacryloylethyldimethylbetaine-methacryloylethyltrimethylammonium chloride-2-hydroxyethyl methacrylate copolymer (polyquaternium-48), and an N,N-dimethylaminoethyl methacrylate diethyl sulfate-N,N-dimethylacrylamide-polyethylene glycol dimethacrylate copolymer (polyquaternium-52); more preferably one or more selected from the group consisting of a methacryloyl ethyl trimethylammonium polymer (polyquaternium-37) and an N,N-dimethylaminoethyl methacrylate diethyl sulfate-N,N-dimethylacrylamide-polyethylene glycol dimethacrylate copolymer (polyquaternium-52).

<9>

The method of treating hair according to any one of <1> to <8>, wherein the content of component (A) in the composition I is 0.01% by mass or more and 3.0% by mass or less, preferably 0.02% by mass or more and 2.5% by mass or less, more preferably 0.05% by mass or more and 2.0% by mass or less, further preferably 0.1% by mass or more and 1.5% by mass or less, still more preferably 0.2% by mass or more and 1.5% by mass or less, and still more preferably 0.2% by mass or more and 0.5% by mass or less.

<10>

The method of treating hair according to any one of <1> to <9>, wherein the component (B) comprises one or more anionic polymers selected from the group consisting of a cross-linked polymer containing a constituent unit derived from (meth)acrylic acid and anionic polysaccharides.

<11>

The method of treating hair according to <10>, wherein the cross-linked polymer containing a constituent unit derived from (meth)acrylic acid is one or more selected from the group consisting of carboxyvinyl polymer or a salt thereof, (acrylate-alkyl acrylate (C10-30)) cross polymer, (Na acrylate-acryloyl dimethyl taurine-dimethyl acrylamide) cross polymer, and acrylate cross polymer-4.

<12>

The method of treating hair according to <10>, wherein the anionic polysaccharides is a polysaccharide having a carboxy group or a salt thereof, preferably alginic acid, carboxymethyl cellulose, or a salt thereof, and more preferably alginic acid or a salt thereof.

<13>

The method of treating hair according to any one of <1> to <12>, wherein the content of component (B) in the composition I is 0.005% by mass or more and 3.0% by mass or less, preferably 0.01% by mass or more and 2.0% by mass or less, more preferably 0.02% by mass or more and 1.5% by mass or less, further preferably 0.02% by mass or more and 1.0% by mass or less, still more preferably 0.02% by mass or more and 0.5% by mass or less, still more preferably 0.02% by mass or more and 0.3% by mass or less, still more preferably 0.02% by mass or more and 0.2% by mass or less, still more preferably 0.02% by mass or more and 0.1% by mass or less, still more preferably 0.02% by mass or more and 0.1% by mass or less, and still more preferably 0.02% by mass or more and 0.08% by mass or less.

<14>

The method of treating hair according to any one of <1> to <13>, wherein the total content of component (A) and component (B) of the composition I is 0.015% by mass or more and 6.0% by mass or less, preferably 0.02% by mass or more and 5.0% by mass or less, more preferably 0.03% by mass or more and 4.5% by mass or less, further preferably 0.04% by mass or more and 4.0% by mass or less, still more preferably 0.05% by mass or more and 3.8% by mass or less, still more preferably 0.07% by mass or more and 3.5% by mass or less, still more preferably 0.07% by mass or more and 3.5% by mass or less, still more preferably 0.1% by mass or more and 3.0% by mass or less, still more preferably 0.12% by mass or more and 2.5% by mass or less, still more preferably 0.22% by mass or more and 2.0% by mass or less, still more preferably 0.22% by mass or more and 0.8% by mass or less, still more preferably 0.22% by mass or more and 0.7% by mass or less, still more preferably 0.22% by mass or more and 0.6% by mass or less, and still more preferably 0.22% by mass or more and 0.58% by mass or less.

<15>

The method of treating hair according to any one of <1> to <14>, wherein a mass ratio [(A)/(B)] of the component (A) to the component (B) contained in the composition I is 1.0 or more and 20 or less, preferably 2.0 or more and 15 or less, more preferably 3.0 or more and 12 or less, further preferably 4.0 or more and 12 or less, and still more preferably 5.0 or more and 10 or less.

<16>

The method of treating hair according to any one of <1> to <15>, wherein the polyglycerin-modified silicone is one or more selected from the group consisting of a polyglycerin-modified silicone include polyglyceryl-3-polydimethylsiloxyethyl dimethicone, polyglyceryl-3-disiloxane dimethicone, lauryl polyglyceryl-3-polydimethylsiloxyethyl dimethicone, cetyldiglyceryltris (trimethylsiloxy)silylethyldimethicone, and bis(polyglyceryl-3-oxyphenylpropyl)dimethicone.

<17>

The method of treating hair according to any one of <1> to <16>, wherein the amino-modified silicones is a silicone represented by the following general formula (2).

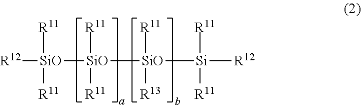

wherein R¹¹ each independently represents a methyl group or a phenyl group, and R¹² each independently represents an alkyl group having 1 or more and 30 or less carbon atoms, a hydroxy group, or R¹³; R¹³ represents a monovalent group represented by —R¹⁴—Z¹ (R¹⁴ represents a single bond or a divalent hydrocarbon group having 1 or more and 20 or less carbon atoms, and Z¹ represents a primary to tertiary amino group-containing group or an ammonium group-containing group); and a is a number of 0 or more and 3000 or less, b is a number of 1 or more and 3000 or less, and a+b is a number of 3,000 or more and 20,000 or less.
<18>

The method of treating hair according to <17>, wherein the amino-modified silicones is one or more selected from the group consisting of aminoethylaminopropyldimethicone [amodimeticone], aminopropyldimethicone, bis(aminopropyl)dimethicone, and bis(cetearyl)amodimethicone.

<19>

The method of treating hair according to any one of <1> to <18>, wherein the content of component (C) in the composition II is 0.01% by mass or more and 5.0% by mass or less, preferably 0.02% by mass or more and 3.0% by mass or less, mor preferably 0.05% by mass or more and 2.0% by mass or less, still more preferably 0.1% by mass or more and 1.0% by mass or less, and still more preferably 0.5% by mass or more and 1.0% by mass or less.

<20>

The method of treating hair according to any one of <1> to <19>, wherein the component (D) is a dimethylpolysiloxane having a viscosity of 300,000 mm²/s or more and 5,000,000 mm²/s or less at 25 °C.

<21>

The method of treating hair according to any one of <1> to <20>, wherein the content of component (D) in the composition II is 0.05% by mass or more and 15% by mass or less, preferably 0.1% by mass or more and 10% by mass or less, more preferably 0.3% by mass or more and 8.0% by mass or less, further preferably 0.5% by mass or more and 5.0% by mass or less, and still more preferably 1.0% by mass or more and 4.0% by mass or less.

<22>

The method of treating hair according to any one of <1> to <21>, wherein the total content of component (C) and component (D) in the composition II is 0.06% by mass or more and 20% by mass or less, preferably 0.1% by mass or more and 15% by mass or less, more preferably 0.3% by mass or more and 10% by mass or less, further preferably 0.5% by mass or more and 10% by mass or less, still more preferably 0.6% by mass or more and 6.0% by mass or less, and still more preferably 1.0% by mass or more and 5.0% by mass or less.

<22>

The method of treating hair according to any one of <1> to <21>, wherein the composition II further comprises (E) a cationic surfactant.

<23>

The method of treating hair according to <22>, wherein the cationic surfactant is one or more selected from the group consisting of (i) alkyltrimethylammonium salt, (ii) alkoxyalkyltrimethylammonium salt, (iii) dialkyldimethylammonium salt, (iv) alkylamide alkyltrimethylammonium salt, (v) alkyldimethylamine and a salt thereof, (vi) alkoxyalkyldimethylamine and a salt thereof, and (vii) alkylamide alkyldimethylamine and a salt thereof; more preferably one or more selected from the group consisting of (i) alkyltrimethylammonium salt and (vi) alkoxyalkyldimethylamine and a salt thereof; more preferably one or more selected from the group consisting of cetyltrimethylammonium chloride, stearyltrimethylammonium chloride, behenyltrimethylammonium chloride, N,N-dimethyl-3-hexadecyloxypropylamine or a lactate thereof, and N,N-dimethyl-3-octadecyloxypropylamine (stearoxypropyldimethylamine) or a lactate thereof; still more preferably one or more selected from the group consisting of behenyltrimethylammonium chloride and N,N-dimethyl-3-octadecyloxypropylamine (stearoxypropyldimethylamine) or a lactate thereof.

<24>

The method of treating hair according to <22> or <23>, wherein the content of component (E) in the composition II is 0.1% by mass or more and 10% by mass or less, preferably 0.2% by mass or more and 5.0% by mass or less, more preferably 0.5% by mass or more and 3.5% by mass or less, and further preferably 2.0% by mass or more and 3.0% by mass or less.

<25>

The method of treating hair according to any one of <1> to <24>, wherein the composition II further comprises (F) higher alcohol.

<26>

The method of treating hair according to <25>, wherein the higher alcohol is one or more selected from the group consisting of cetyl alcohol, myristyl alcohol, stearyl alcohol, and behenyl alcohol.

<27>

The method of treating hair according to <25> or <26>, wherein content of component (F) in the composition II is 0.1% by mass or more and 15% by mass or less, preferably 0.2% by mass or more and 12% by mass or less, more preferably 0.5% by mass or more and 12% by mass or less, still more preferably 1.0% by mass or more and 10% by mass or less, and still more preferably 5.0% by mass or more and 10% by mass or less.

<28>

The method of treating hair according to any one of <1> to <27>, wherein the composition I further comprises a nonionic polymer (G).

<29>

The method of treating hair according to <28>, wherein the nonionic polymer (G) is one or more selected from the group consisting of polyalkylene glycol or the derivative thereof and a polymer containing a constituent unit derived from vinylpyrrolidone, preferably one or more selected from the group consisting of polyethylene glycol, polypropylene glycol, polyethylene glycol-polypropylene glycol copolymer, monofatty or difatty acid ester thereof, and polyvinylpyrrolidone, more preferably one or more selected from the group consisting of polyethylene glycol, polyethylene glycol-polypropylene glycol copolymer, monofatty or difatty acid ester thereof, and polyvinylpyrrolidone.

<30>

The method of treating hair according to <28> or <29>, wherein the content of component (G) in the composition I is 0.01% by mass or more and 3.0% by mass or less, preferably 0.02% by mass or more and 2.5% by mass or less, more preferably 0.05% by mass or more and 2.0% by mass or less, still more preferably 0.1% by mass or more and 1.5% by mass or less, further preferably 0.1% by mass or more and 1.0% by mass or less, and still more preferably 0.2% by mass or more and 0.6% by mass or less.

<31>

The method of treating hair according to any one of <1> to <30>, wherein the composition I further comprises (H) acid.

<32>

The method of treating hair according to <31>, wherein the acid is one or more selected from the group consisting of aliphatic dicarboxylic acids, hydroxycarboxylic acids, and aromatic sulfonic acids, preferably one or more selected from the group consisting of succinic acid, lactic acid, malic acid, glycolic acid, p-toluenesulfonic acid, and naphthalenesulfonic acid, and more preferably one or more selected from the group consisting of succinic acid and lactic acid, and further preferably lactic acid.

<33>

The method of treating hair according to <31> or <32>, wherein the content of component (H) in the composition I is 0.01% by mass or more and 5.0% by mass or less, preferably 0.02% by mass or more and 3.0% by mass or less, more preferably 0.03% by mass or more and 3.0% by mass or less, further more preferably 0.05% by mass or more and 3.0% by mass or less, still more preferably 0.05% by mass or more and 3.0% by mass or less, still more preferably 0.1% by mass or more and 3.0% by mass or less, still more preferably 0.1% by mass or more and 2.5% by mass or less, still more preferably 0.2% by mass or more and 2.5% by mass or less, and still more preferably 0.2% by mass or more and 0.5% by mass or less.

<34>

The method of treating hair according to any one of <1> to <33>, wherein the viscosity of the composition I at 30 °C is 1 mPa-s or more and 10,000 mPa-s or less, preferably 5 mPa-s or more and 5,000 mPa-s or less, and more preferably 10 mPa-s or more and 1,500 mPa-s or less.

<35>

The method of treating hair according to any one of <1> to <34>, wherein the viscosity of the composition II at 30 °C is 10,000 mPa-s or more and 200,000 mPa-s or less, preferably 30,000 mPa-s or more and 150,000 mPa-s or less, and more preferably 60,000 mPa-s or more and 120,000 mPa-s or less.

<36>

The method of treating hair according to any one of <1> to <35>, wherein step 1 and step 2 are performed consecutively.

<37>

The method of treating hair according to any one of <1> to <36>, further comprising a post-step wherein composition I and composition II applied to the hair are rinsed with water.

<38>

The method of treating hair according to any one of <1> to <36>, further comprising any one or more steps selected from the following step A and step B.
Step A: a step of applying a shampoo to the hair and optionally rinsing the shampooed hair
Step B: a step of applying a conditioner to the hair and optionally rinsing the conditioner-applied hair
   <39>

A kit for treating hair, comprising composition I comprising the following component (A) and component (B) for application to hair, and composition II comprising the following component (C) and component (D) for application to hair.
Component (A): cationic polymer having a viscosity of 1,000 mPa-s or more at 30 °C in a 1% by mass aqueous solution;
Component (B): anionic polymer;
Component (C): one or more modified silicones selected from the group consisting of polyglycerin-modified silicone and amino-modified silicone; and
Component (D): dimethylpolysiloxane.
   <40>

A kit for treating hair, comprising composition I comprising the following component (A) and component (B) for application to hair, and composition II comprising the following component (C) and component (D) for application to hair, the kit being for use in a method of treating hair wherein composition I and composition II are applied to the hair in any order and not comprising rinsing the hair with water between application of composition I and application of composition II.
Component (A): cationic polymer having a viscosity of 1,000 mPa-s or more at 30 °C in a 1% by mass aqueous solution;
Component (B): anionic polymer;
Component (C): one or more modified silicones selected from the group consisting of polyglycerin-modified silicone and amino-modified silicone; and
Component (D): dimethylpolysiloxane.
   <41>

Use of a combination of composition I comprising the following component (A) and component (B), and composition II comprising the following component (C) and component (D), for improving the shape and texture of hair, preferably not depending on the diameter of the hair and the degree of damage.
Component (A): cationic polymer having a viscosity of 1,000 mPa-s or more at 30 °C in a 1% by mass aqueous solution;
Component (B): anionic polymer;
Component (C): one or more modified silicones selected from the group consisting of polyglycerin-modified silicone and amino-modified silicone; and
Component (D): dimethylpolysiloxane.
   <42>

Use of a combination of composition I comprising the following component (A) and component (B), and composition II comprising the following component (C) and component (D), for sustaining improved shape and texture of hair, preferably not depending on the diameter of the hair and the degree of damage.
Component (A): cationic polymer having a viscosity of 1,000 mPa-s or more at 30 °C in a 1% by mass aqueous solution;
Component (B): anionic polymer;
Component (C): one or more modified silicones selected from the group consisting of polyglycerin-modified silicone and amino-modified silicone; and
Component (D): dimethylpolysiloxane.
   <43>

The use according to <37> or <38>, wherein composition I and composition II are applied to the hair in any order and not comprising rinsing the hair with water between application of composition I and application of composition II.

### EXAMPLES

Hereinafter, the present invention will be described with reference to Examples; however, the present invention is not limited to the scope of the Examples.

### (Measurement of Viscosity of 1% by Mass Aqueous Solution of Cationic Polymer)

A 1% by mass aqueous solution of a cationic polymer (component (A) or component (A′)) used in each example was prepared, and the viscosity of the aqueous solution at 30 °C was measured by using a B-type viscometer (TV-20, manufactured by Toki Sangyo Co., Ltd.). In the present examples, the cationic polymer other than component (A) is referred to as “component (A′)”.

### (Measurement of Viscosity of the Compositions)

The viscosity of the composition I at 30 °C was measured by using a B-type viscometer (TVB-10, manufactured by Toki Sangyo Co., Ltd.) under conditions of spindle “M3”, 12 rpm and 1 minute, and the viscosity of the composition II at 30 °C was measured by using a B-type viscometer (TVB-10, manufactured by Toki Sangyo Co., Ltd.) under conditions of spindle “T-C”, 30 rpm and 1 minute

### Example 1

### (1) Preparation of Hair Tress

Straight hair of Asian women (thin hair type: hair diameter is 40 to 70 mm; also referred to as hair type F) and straight hair of Asian women (thick hair type: hair diameter is 80 to 110 mm; also referred to as hair type T) were used to prepare hair tress with a length of 25 cm and a mass of 5 g, respectively.

### (2) Preparation of Composition I and Composition II

As composition I, the amount of each component shown in Table 1 below was used to prepare composition I-1. As composition II, the amount of each component shown in Table 1 below was used to prepare composition II-1.

TABLE 1

| | | Composition I | | | | | |
|---|---|---|---|---|---|---|---|
| (% by mass) | | I-1 | I-2 | I-3 | I-4 | I-c1 | I-c2 |
| (A) | Polyquaternium-52 *1 | 0.35 | | 0.35 | 0.35 | | 0.35 |
| | Polyquaternium-37 *2 | | 0.35 | | | | |
| (A′) | Polyquaternium-10 *3 | | | | | 0.35 | |
| | Polyquaternium-22 *4 | | | | | | 0.05 |
| (B) | Sodium alginate *5 | 0.05 | 0.05 | | | 0.05 | |
| | Carboxymethyl cellulose *6 | | | 0.05 | | | |
| | (Acrylic acid/stearyl acrylate) | | | | 0.05 | | |
| | copolymer *7 | | | | | | |
| (G) | Polyvinylpyrrolidone *8 | 0.4 | 0.4 | 0.4 | 0.4 | 0.4 | 0.4 |
| (H) | Lactic acid *9 | 0.3 | 0.3 | 0.3 | 0.3 | 0.3 | 0.3 |
| Other | Dipropylene glycol *10 | 10 | 10 | 10 | 10 | 10 | 10 |
| components | Water | Balance | Balance | Balance | Balance | Balance | Balance |

| | | | | | | |
|---|---|---|---|---|---|---|
| Total | 100 | 100 | 100 | 100 | 100 | 100 |
| Viscosity at 30 °C (mPa-s) | 321 | 58 | 620 | 616 | 47 | 254 |

TABLE 2

| | | Composition II | | | |
|---|---|---|---|---|---|
| (% by mass) | | II-1 | II-2 | II-c1 | II-c2 |
| (C) | Amino-modified silicone *11 | | 0.7 | | |
| | Amino-modified silicone *12 | | | | 0.7 |
| (D) | Dimethylpolysiloxane *13 | | 2.7 | 2.7 | |
| Mixture of (C) | Amino-modified silicone | 0.7 | | | |
| and (D) *14 | Dimethylpolysiloxane | 3.3 | | | |
| (E) | Stearoxypropyl | 2.43 | 2.43 | 2.43 | 2.43 |
| | dimethylamine *15 | | | | |
| (F) | Stearyl alcohol *16 | 7.5 | 7.5 | 7.5 | 7.5 |
| Other | Water | Balance | Balance | Balance | Balance |
| components | | | | | |

| | | | | |
|---|---|---|---|---|
| Total | 100 | 100 | 100 | 100 |
| Viscosity at 30 °C (mPa-s) | 110,700 | 95,500 | 82,600 | 93,700 |

The components listed in the table are as follows. In addition, the formulation amount (% by mass) described in the table is an effective amount of each component.

### <Component (A): Cationic Polymer>

1 Polyquaternium-52 (N,N-dimethylaminoethylmethacrylatediethylsulfate-N,N-dimethylacrylamide-polyeth ylene glycol dimethacrylate copolymer), “SOFCARE KG-101W-E” manufactured by Kao Corporation; charge density of 0.83 mmol/g, 1% viscosity of 2,560 mPa-s, amount of active component of 2.4% by mass
2 Polyquaternium-37 (methacryloylethyltrimethylammonium chloride polymer), “Cosmedia Ultragel 300” manufactured by BASF Japan Ltd.; charge density of 4.81 mmol/g, 1% viscosity of 17,650 mPa-s
   <Component (A′): Cationic Polymer Other than Component (A)>
3 Polyquaternium-10 (cationized hydroxyethyl cellulose), “UCARE Polymer JR-400P” manufactured by Dow Chemical Japan Co., Ltd.; 1% viscosity of 200 mPa-s or less
4 Polyquaternium-22 (dimethyldiallyl ammonium chloride-acrylic acid copolymer liquid), “Marcourt 295” manufactured by Lubrizol Advanced Materials, Inc.; charge density of 6.04 mmol/g, 1% viscosity of 5.6 mPa-s, amount of active component of 37% by mass

### <Component (B): Anionic Polymer>

5 Sodium alginate, “Kimika Argin I-3-150” manufactured by Kimika Co., Ltd., Mw: 90000
6 Carboxymethyl cellulose, “Cellogen F-5a” manufactured by Dai-ichi Kogyo Seiyaku Co., Ltd., Mw of 150000
7 Acrylate-C10-30 alkyl acrylate cross polymer, “Pemulen TR-1 Polymer” manufactured by Lubrizol Advanced Materials, Inc.

### <Component (G): Nonionic Polymer>

8 Polyvinylpyrrolidone, “Luviskol K90” manufactured by BASF Japan Ltd., Mw of 1,200,000

### <Component (H): Acid>

9 90% lactic acid, “PURAC ULTRAPURE 90” manufactured by PURAC Thailand Ltd.

### <Other Componets>

10 Dipropylene glycol, “DPG-RF” manufactured by ADEKA CORPORATION

### <Component (C): Modified Silicone>

11 Amino-modified silicone, “DOWSIL 2-8566 AMINO FLUID” manufactured by Dow Toray Co., Ltd.
12 Amino-modified silicone, “Silicone SF 8457 C” manufactured by Dow Toray Co., Ltd.

### <Component (D): Dimethylpolysiloxane>

13 Dimethylpolysiloxane, “Silicone KHS-3” manufactured by Shin-Etsu Chemical Co., Ltd.

### <Component (C): Modified Silicone and Component (D): Dimethylpolysiloxane>

14 Mixture of dimethylpolysiloxane (number average degree of polymerization 550), dimethylpolysiloxane (number average degree of polymerization 2700), and amino-modified silicone (mass ratio 10:3.7:2.9), “DOWSIL CF1046” manufactured by Dow Toray Co., Ltd.

### <Component (E): Cationic Surfactant>

15 Stearoxypropyl dimethylamine, “Farmin DM-E80” manufactured by Kao Corporation, active component amount: 90% by mass

### <Component (F): Higher Alcohol>

16 Stearyl alcohol, “LANETTE 18” manufactured by BASF

### (3) Hair Treatment

### Preliminary Step

The hair tress of hair type F and the hair tress of hair type T produced above were washed with shampoo and rinsed. Then, the hair tress was dried by towel drying until the total mass of the hair tress reached 7.5 g (150% of the mass of the hair tress before washing).

### Step 1

Composition I-1 was applied to each of hair tress of hair type F and hair tress of hair type T such that the ratio between the dry mass of the hair tress and the mass of the composition I-1, i.e. bath ratio, was 1:0.2, and then combing was performed 5 times and the composition I-1 was spread evenly on the hair tress.

### Step 2

Without rinsing the composition I-1 and without any other steps, composition II-1 was applied to the hair tress to which the composition I-1 had been applied in step 1 such that the ratio between the dry mass of the hair tress and the mass of the composition II-1, i.e. bath ratio, was 1:0.2, and then combing was performed 5 times and the composition II-1 was spread evenly on the hair tress.

### Later Step

The hair tress was rinsed with warm water at 35 °C for 30 seconds, and using a hair dryer (trade name “Nobby Professional Protector Ion Hair Dryer NIB3000” manufactured by Tescom Denki Co., Ltd.) from a position 15 cm away from the hair tress, blow drying was performed with warm air while combing for 2 minutes.

### Evaluation Method

Two expert panelists evaluated the hair tress after hair treatment regarding the items of “moisturized feel”, “smoothness”, “softness”, “lightweight effect”, and “natural” by a 5-point scale from 1 (significantly low or significantly inferior) to 5 (significantly high or significantly superior).

Details of the evaluation items are shown below.

Moisturized hair feel was evaluated by the tactile impression of the hair, such as soft hair touch and flexibility in movement of the hair. Since dry hair is characterized by brittle and porous hair, a low feeling of porosity is another indicator for moisturized hair feel.

Smoothness was evaluated by the appearance and touch feeling of the hair surface. Smoothness in hair is associated with an even surface, so the hair appearance of smooth hair is shiny and sleek without frizz or unruly hair fibers sticking out. The touch feeling of smooth hair is even and silky without roughness and tangling.

The softness of hair was evaluated by touching the hair tresses. Soft hair shows no resistance and moves flexibly when touched or combed. The touch feeling can be described as pleasant and natural.

Lightweight effect was evaluated by the appearance and touch feeling of the hair. Volume and bounciness are the most important indicators for a lightweight care effect. With lightweight care, the hair fibers do not show any signs of coating or weigh-down.

The natural state of hair was evaluated by the appearance and touch feeling of the hair tresses. Whereas an unnatural hair feeling can be described as artificial and coated, natural hair feels flexible and light. Natural hair is perceived as healthy and bouncy.

Table 3 shows the average value of the evaluations of the two expert panelists as the evaluation results.

Smoothness and moistness have a trade-off relationship with the evaluation of lightness and naturalness, and in any evaluation, those with a score of 3 or more are well-balanced and are evaluated as having the effect of the present invention sufficiently.

### Example 2

Hair treatment was performed in the same manner as in Example 1, except that composition II-2 was prepared by using the amount of each component shown in Table 2 above as the composition II, and evaluation was performed. The results of the evaluation are shown in Table 3.

### Example 3

Hair treatment was performed in the same manner as in Example 1, except that composition I-2 was prepared by using the amount of each component shown in Table 1 above as the composition I, and evaluation was performed. The results of the evaluation are shown in Table 3.

### Example 4

Hair treatment was performed in the same manner as in Example 1, except that composition I-3 was prepared by using the amount of each component shown in Table 1 above as the composition I, and evaluation was performed. The results of the evaluation are shown in Table 3.

### Example 5

Hair treatment was performed in the same manner as in Example 1, except that composition I-4 was prepared by using the amount of each component shown in Table 1 above as the composition I, and evaluation was performed. The results of the evaluation are shown in Table 3.

### Example 6

### Preliminary Step

The preliminary step was performed in the same manner as in Example 1.

### Step 2

Composition II-1 prepared in the same manner as in Example 1 was applied to each of hair tress of hair type F and hair tress of hair type T such that the ratio between the dry mass of the hair tress and the mass of the composition II-1 was 1:0.2, and then combing was performed 5 times and the composition II-1 was spread evenly on the hair tress.

### Step 1

Without rinsing the composition II-1 and without any other steps, the composition I-1 prepared in the same manner as in Example 1 was applied to the hair tress to which the composition I-1 had been applied in step 1 such that the ratio between the dry mass of the hair tress and the mass of the composition I-1 was 1:0.2, and then combing was performed 5 times and the composition I-1 was spread evenly on the hair tress.

### Later Step

The later step was performed in the same manner as in Example 1.

### Comparative Example 1

Only the composition I-1 was applied to the hair, and the hair was treated in the same manner as in Example 1 for evaluation. The results of the evaluation are shown in Table 3.

### Comparative Example 2

Only the composition II-1 was applied to the hair, and the hair was treated in the same manner as in Example 1 for evaluation. The results of the evaluation are shown in Table 3.

### Comparative Example 3

Hair treatment was performed in the same manner as in Example 1, except that composition I-c1 was prepared by using the amount of each component shown in Table 1 above as the composition I, and evaluation was performed. The results of the evaluation are shown in Table 3.

### Comparative Example 4

Hair treatment was performed in the same manner as in Example 1, except that composition I-c2 was prepared by using the amount of each component shown in Table 1 above as the composition I, and evaluation was performed. The results of the evaluation are shown in Table 3.

### Comparative Example 5

Hair treatment was performed in the same manner as in Example 1, except that composition II-c1 was prepared by using the amount of each component shown in Table 2 above as the composition II, and evaluation was performed. The results of the evaluation are shown in Table 3.

### Comparative Example 6

Hair treatment was performed in the same manner as in Example 1, except that composition II-c2 was prepared by using the amount of each component shown in Table 2 above as the composition II, and evaluation was performed. The results of the evaluation are shown in Table 3.

### Comparative Example 7

Hair treatment was performed in the same manner as in Example 1, except that the hair tress was rinsed with warm water at 35 °C for 30 seconds between step 1 and step 2, and evaluation was performed. The results of the evaluation are shown in Table 3.

### Comparative Example 8

Hair treatment was performed in the same manner as in Example 6, except that the hair tress was rinsed with warm water at 35 °C for 30 seconds between step 2 and step 1, and evaluation was performed. The results of the evaluation are shown in Table 3.

TABLE 3

| Examples | | | | | | Comparative Examples | | | | | | | |
|---|---|---|---|---|---|---|---|---|---|---|---|---|---|
| 1 | 2 | 3 | 4 | 5 | 6 | | | 3 | 4 | 5 | 6 | 7 | 8 |
| I-1 | I-1 | I-2 | I-3 | I-4 | II-1 | 1 | 2 | I-c1 | I-c2 | I-1 | I-1 | I-1 | II-1 |
| ↓ | ↓ | ↓ | ↓ | ↓ | ↓ | Only | Only | ↓ | ↓ | ↓ | ↓ | Rinse | Rinse |
| II-1 | II-2 | II-1 | II-1 | II-1 | I-1 | I-1 | II-1 | II-1 | II-1 | II-c1 | II-c2 | II-1 | I-1 |

Hair Type T

| | | | | | | | | | | | | | | |
|---|---|---|---|---|---|---|---|---|---|---|---|---|---|---|
| Moisturized feel | 5 | 4 | 3 | 5 | 5 | 4 | 2 | 3 | 3 | 3 | 4 | 5 | 3 | 3 |
| (Moisturized | | | | | | | | | | | | | | |
| feel) | | | | | | | | | | | | | | |
| Smoothness | 5 | 5 | 4 | 5 | 5 | 4 | 2 | 2 | 3 | 3 | 2.5 | 4 | 3 | 3 |
| (Smoothness) | | | | | | | | | | | | | | |
| Soft | 5 | 3 | 3 | 5 | 5 | 4 | 1 | 3 | 2.5 | 3 | 4 | 3 | 3 | 3 |
| (Soft) | | | | | | | | | | | | | | |
| Lightweight | 5 | 5 | 4 | 5 | 5 | 4 | 3 | 3 | 3 | 5 | 1.5 | 1.5 | 3 | 3 |
| effect | | | | | | | | | | | | | | |
| (Lightweight | | | | | | | | | | | | | | |
| effect) | | | | | | | | | | | | | | |
| Natural | 5 | 4 | 4 | 5 | 5 | 4 | 2 | 3 | 3 | 3 | 1.5 | 1.5 | 3 | 2.5 |
| (Natural) | | | | | | | | | | | | | | |

Hair Type F

| | | | | | | | | | | | | | | |
|---|---|---|---|---|---|---|---|---|---|---|---|---|---|---|
| Moisturized feel | 5 | 4 | 4 | 5 | 5 | 4 | 2 | 3 | 3 | 3 | 3 | 5 | 3 | 3 |
| (Moisturized | | | | | | | | | | | | | | |
| feel) | | | | | | | | | | | | | | |
| Smoothness | 5 | 4 | 5 | 5 | 5 | 4 | 2 | 3 | 3 | 2.5 | 3 | 3 | 3 | 3 |
| (Smoothness) | | | | | | | | | | | | | | |
| Soft | 5 | 3 | 3 | 5 | 5 | 4 | 2 | 3 | 3 | 3 | 3 | 3 | 3 | 3 |
| (Soft) | | | | | | | | | | | | | | |
| Lightweight | 5 | 5 | 5 | 5 | 5 | 4 | 2 | 3 | 3 | 2.5 | 2 | 1 | 3 | 3 |
| effect | | | | | | | | | | | | | | |
| (Lightweight | | | | | | | | | | | | | | |
| effect) | | | | | | | | | | | | | | |
| Natural | 5 | 4 | 4 | 5 | 5 | 4 | 1.5 | 3 | 3 | 2.5 | 3 | 1 | 3 | 2.5 |
| (Natural) | | | | | | | | | | | | | | |

As shown in Table 3, the method of treating hair of the present invention is found to allow achieving the effect of improving the shape and texture of hair and the sustainability of the effect without depending on the diameter of the hair and the degree of damage, and simultaneously achieving not only smoothness and moistness but also lightness and naturalness.

### INDUSTRIAL APPLICABILITY

The present invention can provide the method of treating hair that can achieve the effect of improving the shape and texture of hair and the sustainability of the effect without depending on the diameter of the hair and the degree of damage, and can simultaneously achieve not only smoothness and moistness but also lightness and naturalness.

1. A method of treating hair, the method comprising, in any order:
applying composition I comprising component (A) and component (B) to the hair:
Component (A): a cationic polymer having a viscosity of 1,000 mPa-s or more at 30 °C in a 1% by mass aqueous solution; and
Component (B): an anionic polymer; and
applying composition II comprising component (C) and component (D) to the hair:
Component (C): one or more modified silicones selected from the group consisting of polyglycerin-modified silicones and amino-modified silicones; and
Component (D): a dimethylpolysiloxane,
wherein the hair is not rinsed with water between the applying composition I and the applying composition II.

2. The method of treating hair according to claim 1, wherein the component (A) is a polymer comprising a constituent unit represented by formula (1): 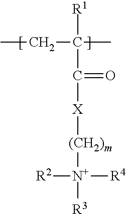
wherein R¹ is a hydrogen atom or a methyl group, R² to R⁴ are each independently an alkyl group having 1 to 4 carbon atoms, X is —O— or —NH—, and m is a number of 1 to 4.

3. The method of treating hair according to claim 1, wherein the component (B) comprises one or more anionic polymers selected from the group consisting of a cross-linked polymer containing a constituent unit derived from (meth)acrylic acid and an anionic polysaccharide.

4. The method of treating hair according to claim 3, wherein the component (B) is alginic acid or a salt thereof.

5. The method of treating hair according to claim 1, wherein the component (A) is one or more selected from the group consisting of a methacryloyl ethyl trimethylammonium chloride polymer and an N,N-dimethylaminoethyl methacrylate diethyl sulfate-N,N-dimethylacrylamide-polyethylene glycol dimethacrylate copolymer.

6. The method of treating hair according to claim 1, wherein the component (D) is a dimethylpolysiloxane having a viscosity of from 300,000 mm²/s to 5,000,000 mm²/s at 25 °C.

7. The method of treating hair according to claim 1, wherein a mass ratio [(A)/(B)] of the component (A) to the component (B) contained in the composition I is from 1.0 to 20.

8. The method of treating hair according to claim 1, wherein the composition I further comprises a nonionic polymer (G).

9. The method of treating hair according to claim 8, wherein the nonionic polymer (G) is one or more selected from the group consisting of a polyalkylene glycol or a derivative thereof and a polymer containing a constituent unit derived from vinylpyrrolidone.

10. The method of treating hair according to claim 1, wherein the applying composition I and the applying composition II are performed consecutively.

11. A kit for treating hair, comprising:
composition I for application to hair, comprising:
Component (A): a cationic polymer having a viscosity of 1,000 mPa-s or more at 30 °C in a 1% by mass aqueous solution; and
Component (B): an anionic polymer; and
composition II for application to hair, comprising:
Component (C): one or more modified silicones selected from the group consisting of polyglycerin-modified silicone and amino-modified silicone; and
Component (D): dimethylpolysiloxane.

12. The method of treating hair according to claim 1, wherein the composition I is a hair conditioner, a hair treatment, or a hair styling agent.

13. The method of treating hair according to claim 1, wherein the composition II is a hair treatment or a hair styling agent.

14. The method of treating hair according to claim 1, wherein the component (C) is an amino-modified silicone.

15. The kit according to claim 11, wherein the composition I is a hair conditioner, a hair treatment, or a hair styling agent.

16. The kit according to claim 11, wherein the component (A) is one or more selected from the group consisting of a methacryloyl ethyl trimethylammonium chloride polymer and an N,N-dimethylaminoethyl methacrylate diethyl sulfate-N,N-dimethylacrylamide-polyethylene glycol dimethacrylate copolymer.

17. The kit according to claim 11, wherein the content of component (B) in the composition I is from 0.02% by mass to 0.1% by mass.

18. The kit according to claim 11, wherein a mass ratio [(A)/(B)] of component (A) to component (B) contained in the composition I is from 3.0 to 12 or less.

19. The kit according to claim 11, wherein the composition II is a hair treatment or a hair styling agent.

20. The kit according to claim 11, wherein the component (C) is an amino-modified silicone.
